(19) United States
(12) Patent Application Publication
(10) Pub. No.: US 2022/0280469 A1
(43) Pub. Date: Sep. 8, 2022

(54) PHARMACEUTICAL COMPOUND AND PREPARATION METHOD THEREFOR AND USE THEREOF

(71) Applicant: NOVAGENESIS THERAPEUTIX (HK) LIMITED, Hong Kong (CN)

(72) Inventors: Feng QIAN, Beijing (CN); Junxiao YE, Beijing (CN); Chao KONG, Beijing (CN)

(21) Appl. No.: 17/424,622

(22) PCT Filed: Jan. 22, 2020
(86) PCT No.: PCT/CN2020/073666

§ 371 Date: Jan. 13, 2022

(30) Foreign Application Priority Data
Jan. 23, 2019 (CN) 201910064809.3

(51) Int. Cl. A61K 31/282; A61K 47/42; A61P 35/00

(52) U.S. Cl. CPC A61K 31/282; A61K 47/42; A61P 35/00

(57) ABSTRACT

Disclosed are a drug-carrier complex and a preparation method therefor and use thereof. The drug-carrier complex comprises an active ingredient and a carrier, wherein the carrier is albumin, the active ingredient is loaded on the carrier, and the active ingredient is a platinum-based drug. Compared with free platinum-based drugs, the drug-carrier complex can significantly extend in vivo half-life of the platinum-based drug, improve in vivo tissue distribution of the platinum-based drug, increase tissue selectivity of the platinum-based drug, reduce damage to normal tissues and systemic toxicity of the platinum-based drug, and thus improve therapeutic effect.

### TECHNICAL FIELD

The present invention relates to a drug-carrier complex comprising an active ingredient and a carrier, wherein the active ingredient is loaded on the carrier, the active ingredient is a platinum-based drug, and the carrier is albumin. The present invention provides a method for preparing the drug-carrier complex and use of the drug-carrier complex, particularly in the treatment of cancer.

### BACKGROUND

Tumor is one of the major diseases seriously harming human life and health and is characterized by overproliferation and abnormal differentiation of cells. According to statistics from WHO, cancer caused about 8.6 million deaths in 2014. In China, about 2.2 million people died from cancer that year. It is expected that by 2034, the number of cancer cases may increase to 24 million annually worldwide. At the same time, cancer imposes a huge burden on the global economy. Annual cost for cancer was about 1.16 trillion US dollars in 2010. Statistics show that the following cancers are leading causes of human death worldwide: tracheocarcinoma, bronchogenic carcinoma, lung cancer; liver cancer, gastric cancer, esophageal cancer, colorectal cancer and cancers of the reproductive system, namely prostatic cancer, breast cancer and cervical cancer. Treatment of cancer with chemical drugs (abbreviated as “chemotherapy”) is a common method used in treating cancer at present.

Platinum-based drugs are chemotherapeutic agents and are widely used in chemotherapy. Clinically, effective platinum-based drugs are selected for different cancer patients to inhibit the growth of tumor cells or allow the tumor cells to trigger self-death mechanisms, thus curing the cancer. Therefore, clear understanding of the action mechanism of the platinum-based drugs can guide the design of novel platinum-based drugs with high therapeutic activity and improve the clinical therapeutic effect, which is of great significance for the continuation of life.

Platinum-based anti-cancer drugs that have been currently approved for marketing include: cisplatin (cis-[PtCl₂(NH₃)_{2]}, CDDP), carboplatin and oxaliplatin, as well as such drugs approved in certain countries as nedaplatin (China), lobaplatin (Japan), and heptaplatin (South Korea). The platinum-based drugs are clinically used for various solid tumors, including ovarian cancer, prostatic cancer, testicular cancer, lung cancer, nasopharyngeal carcinoma, esophageal cancer, malignant lymphoma, head and neck cancer, thyroid cancer, osteogenic sarcoma and the like. Clinically, the platinum-based drugs can also be used in combination with bleomycin, paclitaxel, 5-fluorouracil, etc., to achieve better therapeutic effect.

Among these platinum-based drugs, cisplatin and similar platinum-based drugs have significant anti-cancer activity in treating a series of malignant tumors. For example, cisplatin is one of the most widely used anti-cancer drugs for treating testicular cancer, ovarian cancer, cervical cancer, bladder cancer, osteosarcoma, head and neck cancer, small cell and non-small cell lung cancer, melanoma, lymphoma, lung cancer, etc., and is also currently used as one of the traditional second-line chemotherapy drugs for pancreatic cancer. In addition, there has been some research progress in the treatment of head and neck squamous carcinoma with cisplatin in combination with a PD-1/PD-L1 inhibitor.

Although more than 50% of cancer patients have been treated with platinum-based chemotherapeutic drugs, these compounds still have serious side effects. Clinical drawbacks of platinum-based drugs (such as cisplatin) mainly include: 1) some platinum-based drugs are absorbed very rapidly upon intravenous injection; after injection, the drug, instead of being distributed in specific sites, is widely distributed in liver, kidney, prostate, bladder and ovary, and can also be found in chest and abdominal cavity; 2) some platinum-based drugs are non-specific cytotoxic drugs, which kill both tumor cells and normal cells, resulting in some serious side effects, such as renal function impairment, liver function impairment, hematopoietic system toxicity, digestive system toxicity, nervous system toxicity and ototoxicity; and 3) some platinum-based drugs have a short in vivo half-life.

Moreover, as chemotherapy process proceeds, the tumor may exhibit acquired resistance that renders the tumor tissue insensitive to the chemotherapeutic drug, resulting in drug resistance and ultimately failure of chemotherapy. The reasons for the development of drug resistance may be the following three: (1) the uptake of platinum-based drugs by cells is insufficient; (2) small molecules and proteins which are metabolized in cells and rich in —NH₂ and —SH can be in complexation with platinum-based drugs, so that the platinum-based drugs cannot bind to DNA to play a role; and (3) after the platinum-based drug and DNA form an adduct, the repair protein can recognize the damaged site and complete the repair of the DNA (see Stewart et al., Onco. & Hema., 63:12-31 (2007)). Of these, the intracellular accumulation of platinum is the major concern. Drug-resistant cells tend to express, on the cell membrane, relatively little Ctrl protein (see Ishida et al., Proc. Nat. Acad. Sci., 99:14298-14302 (2002)), a main transporter for entry of platinum-based drugs into cells, and the reduction in the expression of the protein leads to the reduction of the uptake of the platinum-based drugs by drug-resistant tumor cells, thereby causing less DNAs in the cells to be damaged by the platinum-based drugs.

To increase cellular uptake of platinum-based drugs, it has been reported that nanotechnology is introduced into this chemotherapy process (see Sinha et al., Mol. Can. Ther., 5:1909-1917 (2006)), and organic block polymers, carbon nanotubes and gold nanocrystals are all used as carriers of platinum-based drugs for drug delivery into cells from the outside. However, there's biological safety concern for these nanomaterials. These materials are often nondegradable under physiological conditions, and they themselves may have the ability to destroy normal cells, thereby causing great side effects.

Therefore, there has been ongoing research on modification and alteration of platinum-based drugs. However, at present, effective treatment methods for drug-resistant tumors are still not many, and there's still a need to develop platinum-based drug preparations with better targeting property and higher safety.

### SUMMARY

In one aspect, the present invention provides a drug-carrier complex comprising an active ingredient and a carrier, wherein the active ingredient is loaded on the carrier, the active ingredient is a platinum-based drug, and the carrier is albumin.

In a second aspect, the present invention provides a method for preparing the pharmaceutical composition, which comprises: subjecting the platinum-based drug and the albumin to a mixing-combining process in a specific buffer to obtain a mixture; and purifying the resulting mixture to obtain the drug-carrier complex.

In a third aspect, the present invention provides a pharmaceutical composition comprising the drug-carrier complex and at least one pharmaceutically acceptable carrier.

In a fourth aspect, the present invention provides use of the drug-carrier complex in preparing a medicament for treating cancer.

### BRIEF DESCRIPTION OF THE DRAWINGS

FIG. 1 shows a process flow chart of a cisplatin-albumin compound according to an embodiment of the present invention.

FIG. 2 shows the morphology and structure characterization of a cisplatin-albumin compound according to an embodiment of the present invention, wherein FIG. 2A shows an FPLC spectrum of the purification of cisplatin-albumin compound, and what is shown by HSA-Cis is the sample peak (absorption peak at UV 280) of cisplatin-albumin compound; FIG. 2B shows the dry powder of lyophilized cisplatin-albumin compound and the cisplatin-albumin compound redissolved with PBS solution; FIG. 2C shows the particle size of the cisplatin-albumin compound; FIG. 2D shows SDS-PAGE electropherogram of albumin and cisplatin-albumin compounds (HSA-Cis 1:1 and HSA-Cis 1:6), indicating that the compounds exist primarily in the form of a single protein molecule; FIG. 2E shows the results of mass spectrometry analysis of the cisplatin-albumin compounds, with drug protein ratio (DPR) of HSA-Cis (1:1) being 0.91, HSA-Cis (1:6) being 5.43 and HSA-Cis (1:12) being 11.2.

FIG. 3 shows the performance comparison of cisplatin-albumin compounds prepared at different feed ratios according to an embodiment of the present invention, wherein FIG. 3A shows the change trend of the DPR with the feed ratio of cisplatin; FIG. 3B shows the particle size of cisplatin-albumin compounds with different feed ratios.

FIG. 4 shows tumor cytotoxicity and cell migration inhibition effect of cisplatin-albumin compounds according to an embodiment of the present invention, wherein FIG. 4A shows that cisplatin-albumin compounds with different DPRs have different cytotoxicity to human pancreatic cancer cell strain MIA PaCa-2, and the cell necrosis is induced due to high protein concentration in a low DPR system, and the difference among higher DPR compounds is not significant (n=5); FIG. 4B shows that the cytotoxicity of the cisplatin-albumin compound to human pancreatic cancer cell strains MIA PaCa-2 cells and PANC-1 is lower than that of cisplatin (n=5) due to the difference of the cellular entry pathways of cisplatin and the compound; FIG. 4C shows the cell migration experiment for cisplatin-albumin compounds with different DPRs, with the migration rate of tumor cells treated with cisplatin-albumin compound 6:1 group being the lowest; FIG. 4D shows the quantitative analysis of cell migration experiment.

FIG. 5 shows the pharmacokinetic profiles of cisplatin and cisplatin-albumin compound (HSA-Cis) in plasma according to an embodiment of the present invention.

FIG. 6 shows the pharmacokinetic profiles of cisplatin and cisplatin- albumin compound (HSA-Cis) at tumor sites in pancreatic cancer orthotopic transplantation models according to an embodiment of the present invention.

FIG. 7 shows the effect of cisplatin-albumin compounds with different DPRs in inhibiting tumor volume growth of PANC-1 subcutaneous transplantation tumor models after being administered 4 times via tail vein injection according to an embodiment of the present invention, wherein FIG. 7A shows the tumor volume change curve of human pancreatic cancer cell strain PANC-1 subcutaneous transplantation tumor model; FIG. 7B shows the findings in tumor weight: the HSA-Cis 1:6 compound has the most significant tumor inhibition effect (n=5); FIG. 7C shows change trend of body weight of mice after administration, and it can be seen that the body weight loss after cisplatin administration is significant, indicating that cisplatin has very strong systemic toxicity (n=5) (*P<0.05, **P<0.01, ***P<0.001, ****P<0.0001, *****P<0.00001, two-tailed t-test), and the date marked with the arrow as the administration date; FIG. 7D shows pathological and histochemical analysis of the liver and tumors of mice after the end of the administration cycle, namely HE staining analysis of tumor tissues and liver, Ki67 staining of tumor tissues and TUNEL analysis of tumor tissues.

FIG. 8 shows the evaluation of the therapeutic effect of the cisplatin-albumin compound on the MIA PaCa-2 pancreas orthotopic transplantation models according to an embodiment of the present invention, wherein FIG. 8A shows that the survival advantage of mice in the cisplatin-albumin compound groups, especially in the low dose compound group, is significant after the compound is administered via tail veins; FIG. 8B shows the curves of body weight change of mice after administration, indicating that the compound is less toxic than cisplatin.

FIG. 9 shows the maximum tolerated doses and toxicity evaluation of cisplatin-albumin compounds in vivo according to an embodiment of the present invention, wherein FIG. 9A shows the survival state curves of animals during experiments on maximum tolerated doses of cisplatin-albumin compound and cisplatin (X represents animal death); FIGS. 9B-9E show the results of blood cell analysis 11 days after administration of cisplatin-albumin compounds and cisplatin; FIG. 9F shows the blood biochemical analysis results for liver function 11 days after administration of cisplatin-albumin compounds and cisplatin; FIG. 9G shows blood biochemical analysis results for renal function 11 days after administration of cisplatin-albumin compounds and cisplatin (*P<0.05, **P<0.01, ***P<0.001, ****P<0.0001, *****P<0.00001, two-tailed t-test).

FIG. 10 shows the results of structural characterization and morphological characterization of carboplatin-, nedaplatin- and oxaliplatin-albumin compounds according to an embodiment of the present invention, wherein FIG. 10A shows the results of mass spectrometry analysis of human serum albumin and carboplatin-, nedaplatin-, oxaliplatin- and lobaplatin-albumin compounds; FIG. 10B shows the dry powder of lyophilized oxaliplatin-albumin compound and the oxaliplatin-albumin compound redissolved with saline solution solution; FIG. 10C shows the particle size of oxaliplatin-albumin compounds prepared at different feed ratios; FIG. 10D shows tumor cytotoxicity of oxaliplatin-albumin compound for pancreatic cancer cell strain PANC-1 and colon cancer cell strain DLD-2 according to an embodiment of the present invention; FIG. 10E shows the effect of oxaliplatin-albumin compound prepared according to an embodiment of the present invention and a commercially available oxaliplatin preparation of the same dose in inhibiting tumor proliferation of Panc-1 subcutaneous transplantation tumor models after being administered 6 times via tail vein injection, and shows the change in body weight of mice after administration; FIG. 10F shows the blood cell change in mice 3 days after the administration of the same doses of a commercially available oxaliplatin preparation and the oxaliplatin-albumin compound according to an embodiment of the present invention, and shows the maximum tolerated dose results in vivo after 4 consecutive administrations (twice per week).

### DETAILED DESCRIPTION

After reading of the detailed description, how to implement the present invention in various alternative embodiments and alternative applications will be apparent to those skilled in the art. However, not all the various embodiments of the present invention will be described herein. It should be understood that the embodiments provided herein are presented by way of examples only and are not intended to be limiting. As such, detailed description of various alternative embodiments should not be construed as limiting the scope or breadth of the present invention as set forth below.

Before the present invention is disclosed and described, it should be understood that the aspects described below are not limited to specific compositions, methods for preparing the compositions or uses thereof, and that the aspects described below can, of course, vary. It should also be understood that the terms used herein are for the purpose of describing particular aspects only and are not intended to be limiting.

### Definitions

Unless otherwise stated, all technical and scientific terms used herein have the same meaning as that commonly understood by those of ordinary skill in the art to which the present invention belongs. In the specification and the appended claims, reference will be made to multiple terms which shall be defined to have the following meanings.

The terms used herein are for the purpose of describing particular embodiments only and are not intended to limiting the present invention. The singular forms used herein are also intended to include the plural forms, unless otherwise expressly stated.

“Comprise” or “comprising” means that the compositions and methods comprise the recited ingredients but do not exclude other ingredients. “Consisting essentially of . . . ”, when used to define compositions and methods, means excluding other ingredients that are of any essential significance to the combination for the stated purpose. Thus, a composition consisting essentially of components as defined herein would not exclude other materials or steps that do not materially affect the basic and novel characteristics of the claimed invention. “Consisting of” means excluding more than trace elements of other ingredients and substantial method steps. Embodiments defined by each of these transition terms are within the scope of the present invention.

Unless otherwise stated, the term “at least” preceding a series of elements is to be understood as including all the elements in the series. The terms “at least one” and “at least one of . . . ” include, for example, one, two, three, four or five or more elements. Further, it should be understood that slight variations above and below the stated ranges can be used to achieve substantially the same results as values within the ranges. Also, unless otherwise stated, the disclosure of ranges is intended as a continuous range including every value between the minimum and maximum values.

Platinum-based drugs are alkylating agents that covalently bind to DNA and cross-link DNA strands, resulting in inhibition of DNA synthesis and function and inhibition of transcription. In some embodiments, the platinum-based drug is selected from cisplatin, carboplatin, oxaliplatin, nedaplatin, lobaplatin, picoplatin, satraplatin and triplatin. In some embodiments, the platinum-based agent is carboplatin. In some embodiments, the platinum-based agent is cisplatin.

Cisplatin, namely cis-diamminedichloroplatinum, is commercially available as an injection PLATINOL®. Cisplatin is mainly indicated for the treatment of metastatic testicular cancer and ovarian cancer and advanced bladder cancer. The major dose limiting side effects of cisplatin are toxic renal damage, which can be controlled by hydration and diuresis, and ototoxicity.

Carboplatin, namely diammine[1,1-cyclobutane-dihydroxy acid(2-)-O,O′]platinum, is commercially available as an injection PARAPLATIN®. Carboplatin is indicated for first-line and second-line treatment of advanced ovarian cancer. Myelosuppression is dose limiting toxicity of carboplatin.

“Albumin” is a spherical serum protein with a molecular weight of about 65 kDa. The albumin in the present application can be selected from serum-derived albumins, such as human serum albumin and bovine serum albumin; bioengineered recombinant albumin s, for example, ovalbumin crosslinked by aliphatic dialdehydes, such as glutaraldehyde, glyoxal, dimethylglyoxal, or ketones (e.g. 2,3-butanedione), esters (e.g. ethylene glycol bis(succinimidyl-succinimidyl-succinate)), acid chlorides (e.g. terephthalic acid dichloride) and diisocyanates (e.g. toluene diisocyanate), or by divalent, trivalent and tetravalent metal cations, or by heating (90-170° C., 10-60 min); and analogs thereof (see, Tomlinson et al., “Monolithic albumin particles as drug carrier systems” in Polymers in Control Drug Delivery, Illum et al. (eds.), Wright, Bristol, 1987, p 25-48).

In some embodiments, the albumin is selected from serum-derived albumins, bioengineered recombinant albumin s and analogs thereof. In another embodiment, the serum-derived albumin is selected from human serum albumin and bovine serum albumin.

The amount of albumin contained in the drug-carrier complex disclosed herein will vary depending on the pharmaceutically active agent, other excipients and the route and site of intended administration. The amount of albumin contained in the composition is desirably an amount effective to reduce one or more side effects of the active agent caused by administration of the drug-carrier complex to a human.

According to an embodiment of the present invention, the platinum-based drug and the albumin are linked to form the drug-carrier complex by a manner including, but not limited to, a covalent bond, a coordination bond and a non-covalent bond. In some embodiments, the non-covalent bond includes Van der Waals' force, a hydrogen bond, hydrophobic interaction, and the like.

In some embodiments, an average particle size of the drug-carrier complex is 3-10 nm. After measurement with a dynamic light scattering particle size analyzer, it is found that the average particle size of the drug-carrier complex is 3-10 nm, which is the same as the particle size of albumin. This shows that the platinum-based drug-albumin compound exists in the form of a protein monomer.

In some embodiments, a molar ratio of the platinum-based drug to the albumin in the drug-carrier complex is 0.5:1-24:1, e.g., 0.9:1-14.5:1, such as 0.91, 1.0, 1.5, 2.0, 2.5, 2.65, 2.7, 3.0, 3.5, 4.0, 4.5, 5.0, 5.4, 5.43, 5.5, 6.0, 7.0, 7.3, 7.5, 7.7, 8.0, 9.0, 10.0, 11.0, 11.2, 11.5, 12.0, 13.0, 13.7, 14.0 or 14.5. Within this range, the drug-carrier complex has better therapeutic effect and targeting property at the tumor site, and the drug safety of the drug-carrier complex is higher.

In some embodiments, a molar ratio of the platinum-based drug to the albumin in the drug-carrier complex is 2.5:1-11.2:1, such as 2.6, 2.65, 2.7, 3.0, 3.5, 4.0, 4.5, 5.0, 5.4, 5.43, 5.5, 6.0, 7.0, 7.3, 7.5, 7.7, 8.0, 9.0, 10.0, 11.0 or 11.2. In the drug-carrier complex, if the concentration of the albumin is too high, the osmotic pressure inside and outside cells is easily changed to cause cell necrosis, resulting in higher cytotoxicity; if the concentration of the platinum-based drug is too high, the effect of inhibiting tumor cell proliferation is no longer increased with the increase in the concentration of the platinum-based drug, and other normal tissues may be damaged as well due to the high concentration of the platinum-based drug. Within this range, the therapeutic effect and toxicity of the drug-carrier complex are in a balanced level, and the drug effect can be effectively exerted while the toxicity can be effectively reduced.

In some embodiments, a molar ratio of the platinum-based drug to the albumin in the drug-carrier complex is 5.0:1-7.0:1, such as 5.1, 5.2, 5.3, 5.4, 5.43, 5.5, 5.6, 5.7, 5.8, 5.9, 6.0, 6.1, 6.2, 6.3, 6.4, 6.5, 6.6, 6.7, 6.8 or 6.9. Within this range, the drug-carrier complex has optimal comprehensive effect of the therapeutic effect and targeting property at the tumor site and the drug safety.

In a second aspect of the present invention, the present invention provides a method for preparing the drug-carrier complex described above. According to an embodiment of the present invention, the method comprises: subjecting the platinum-based drug and the albumin to a mixing-combining process in a specific buffer to obtain a mixture; and purifying the resulting mixture to obtain the drug-carrier complex.

In some embodiments, the method further comprises dispersing the platinum-based drug in a solvent prior to mixing-combining. In some embodiments, the solvent is saline solution. In some embodiments, the solvent is glucose injection. In some embodiments, the solvent is water for injection. In some embodiments, the solvent is phosphate buffer. In some embodiments, the solvent is dimethyl sulfoxide. In some embodiments, the solvent is methanol. In some embodiments, the solvent is N,N-dimethylformamide.

In other embodiments, a concentration of the platinum-based drug in saline solution is 0.1-20 mg/mL, e.g., 0.1-5 mg/mL, such as 0.1, 0.2, 0.3, 0.4, 0.5, 1.0, 1.5, 2.0, 2.5, 3.0, 3.5, 4.0 or 4.5 mg/mL.

The term “buffer” includes those agents that maintain the pH of the solution within an acceptable range and may include succinate (e.g., sodium succinate or potassium succinate), histidine, phosphate (e.g., sodium phosphate or potassium phosphate), Tris (tri(hydroxymethyl)aminomethane), diethanolamine, citrate (e.g., sodium citrate), and the like. A pH of the buffers of the present invention is in the range of about 4 to about 10. Examples of buffers that control pH within this range include succinate (e.g., sodium succinate), gluconate, histidine, citrate, other organic acid buffers, and the like.

In some embodiments, a pH of the buffer is 5.0-7.5, such as 5.0, 5.3, 5.5, 5.7, 6.0, 6.3, 6.5, 6.7, 7.0, 7.1, 7.2, 7.3 or 7.4.

In some embodiments, the buffer comprises 20-60 mmol/L, such as 30, 35, 40, 45, 50 or 55 mmol/L phosphate. In some embodiments, the buffer comprises 10-200 mmol/L, such as 20, 30, 40, 50, 60, 70, 80, 90, 100, 110, 120, 130, 140, 150, 160, 170, 180 or 190 mmol/L NaCl. In some embodiments, the buffer comprises 2-20 mmol/L, such as 3, 4, 5, 6, 7, 8, 9, 10, 11, 12, 13, 14, 15, 16, 17, 18 or 19 mmol/L EDTA. In some embodiments, the buffer comprises 20-60 mmol/L phosphate, 10-200 mmol/L NaCl and 2-20 mmol/L EDTA.

In some embodiments, the albumin is dispersed in the buffer at a concentration of 5-50 mg/mL, such as 7, 9, 10, 15, 20, 25, 30, 35, 40 or 45 mg/mL.

In some embodiments, the mixing-combining is performed by adding a dispersion of the platinum-based drug dropwise to a buffer solution of the albumin. Under the condition, the efficiency of preparing the drug-carrier complex is further improved, and the proportion of protein monomer in the drug-carrier complex can be effectively improved.

In some embodiments, a molar ratio of the platinum-based drug to the albumin used is 1:1-1:30, such as 1:2, 1:3, 1:4, 1:5, 1:6, 1:7, 1:8, 1:9, 1:10, 1:11, 1:12, 1:15, 1:20, 1:24 or 1:30. Within this range, the prepared drug-carrier complex has better therapeutic effect and targeting property at the tumor site, and the drug safety is higher.

In some embodiments, a molar ratio of the platinum-based drug to the albumin used is 1:3-1:8, such as 1:4, 1:5, 1:6 or 1:7. If the dose of the albumin is too high, the concentration of the albumin in the prepared drug-carrier complex is too high, and the osmotic pressure inside and outside cells is easily changed to cause cell necrosis when the drug-carrier complex enters into the body, and namely, the drug-carrier complex has higher cytotoxicity; if the dose of the platinum-based drug is too high, the concentration of the platinum-based drug in the prepared drug-carrier complex is too high, and other normal tissues would be very likely to be damaged by the drug-carrier complex while the effect of inhibiting tumor cell proliferation would not be increased significantly with the increase in the concentration of the platinum-based drug. Within this range, the prepared drug-carrier complex has balanced therapeutic effect and toxicity, namely, effectively exerting the drug effect while effectively reducing the toxicity.

In some embodiments, a molar ratio of the platinum-based drug to the albumin used is 1:6. Under this condition, the drug-carrier complex prepared according to the method provided by the embodiment of the present invention has optimal comprehensive performance of therapeutic effect and targeting property at tumor sites and drug safety.

In some embodiments, the mixing-combining is performed at a rotation speed of 300-500 rpm, such as 350, 400,or 450 rpm, for 4-24 hours, such as 4, 5, 6, 7, 8, 9, 10, 11, 12, 13, 14, 16, 18, 20, 22 or 24 hours. Within this range, the efficiency of preparing the drug-carrier complex is further improved, and the particle size of the prepared drug-carrier complex is more uniform.

As used herein, the terms “administer” and “administration” refer to introducing a compound or a composition (e.g., a therapeutic agent) into a mammal in any manner to prevent or treat a disease or disorder (e.g., cancer).

As used herein, the term “cancer” refers to a proliferative disease caused by or characterized by cell proliferation featuring loss of sensitivity to normal growth control. The term “cancer” used herein includes tumors and any other proliferative disease. Cancers of the same tissue type originate in the same tissue and can be divided into different subtypes based on their biological characteristics. The cancer can be selected from, for example, glioblastoma, squamous cell carcinoma, skin cancer-related tumors, breast cancer, head and neck cancer, gynecological cancer, urinary and male genital cancer, bladder cancer, prostatic cancer, bone cancer, endocrine adenocarcinoma, digestive tract cancer, major digestive/organ cancer, central nervous system cancer and lung cancer.

As used herein, “treatment” “treat” or “treating” is a method for obtaining beneficial or desired results, including clinical results. For purposes of the present invention, beneficial or desired clinical results include, but are not limited to, one or more of the following: alleviating one or more symptoms resulting from the disease, reducing the severity of the disease, stabilizing the disease (e.g., preventing or delaying the worsening of the disease), preventing or delaying the spread of the disease (e.g., metastasis), preventing or delaying the recurrence of the disease, delaying or slowing the progression of the disease, ameliorating the disease state, providing disease response (partial or total), reducing the dose of one or more other drugs required to treat the disease, delaying the progression of the disease, improving the quality of life, and/or prolonging survival time. “Treatment” “treat” or “treating” also includes alleviating the pathological consequences of cancer. The method disclosed herein contemplates any one or more of these treatment aspects. “Treatment” of cancer includes, for example, surgery, chemotherapy, radiotherapy, gene therapy and immunotherapy.

“Pharmaceutically effective amount” includes an amount sufficient to ameliorate or prevent a symptom or a sign of a medical disorder. Pharmaceutically effective amount also refers to an amount sufficient to allow or facilitate diagnosis. The effective amount for a particular patient or veterinary object may vary depending on factors such as the disease to be treated, the general health of the patient, the route of method, the dose of administration, and the severity of side effects. The pharmaceutically effective amount may be the maximum dose or administration regimen that avoids significant side effects or toxic effects. The effect will result in an improvement of the diagnostic measure or parameter by at least 5%, such as at least 10%, further such as at least 20%, further such as at least 30%, further such as at least 40%, further such as at least 50%, further such as at least 60%, further such as at least 70%, further such as at least 80%, and even further such as at least 90%, wherein 100% is defined as the diagnostic parameter displayed by a normal subject. According to an embodiment of the present invention, the pharmaceutically effective amount of the platinum-based drug-albumin drug-carrier complex will be an amount, for example, sufficient to reduce tumor volume, inhibit tumor growth or prevent or reduce metastasis.

The term “pharmaceutically acceptable” refers to those compounds, materials, compositions and/or dosage forms which are suitable for being in contact with the tissues of human beings and animals without excessive toxicity, irritation, allergic response, or other problems or complications, and are commensurate with a reasonable benefit/risk ratio. For example, a pharmaceutically acceptable substance may be incorporated into a pharmaceutical composition administered to a patient without causing any significant undesirable biological effects or interacting in a deleterious manner with any of the other components of the composition in which it is contained. The pharmaceutically acceptable carrier or excipient preferably meets the requisite toxicological and manufacturing test standards and/or is included in the Inactive Ingredient Guide provided by U.S. Food and Drug Administration.

The pharmaceutical composition disclosed herein may also comprise other conventional pharmaceutically acceptable ingredients, commonly referred to as carriers, excipients or adjuvants. Examples of such carriers, excipients or adjuvants include, but are not limited to: disintegrants, binders, lubricants, glidants, stabilizers, fillers, diluents, colorants, flavoring agents and preservatives. Those of ordinary skill in the art may, by conventional experimentation, select one or more of the above carriers based on the particular desired properties of the dosage form without undue burden. The amount of each carrier used is within the conventional range in the art. The following documents, incorporated herein by reference, disclose techniques and excipients for formulating oral dosage forms. See, e.g., The Handbook of Pharmaceutical Excipients, 4th edition, ed. by Rowe et al., American Pharmaceuticals Association (2003); and Remington: the Science and Practice of Pharmacy, 20th edition, Gennaro (ed.), Lippincott Williams & Wilkins (2003).

The term “pharmaceutically acceptable carrier” as used herein includes any and all solvents, dispersion media, coating agents, surfactants, antioxidants, preservatives (e.g., antibacterial agents or antifungal agents), isotonic agents, absorption delaying agents, salts, preservatives, drugs, drug stabilizers, binders, excipients, disintegrants, lubricants, sweeteners, flavoring agents, dyes, and the like, and combinations thereof, as known to those skilled in the art (see, e.g., Remington's Pharmaceutical Sciences, 18th edition, Mack Printing Company, 1990, 1289-1329). The use of any conventional carrier in therapeutic or pharmaceutical compositions is contemplated unless it is incompatible with the active ingredient.

Examples of pharmaceutically acceptable antioxidants include: (1) water-soluble antioxidants, such as ascorbic acid, cysteine hydrochloride, sodium bisulfate, sodium metabisulfite and sodium sulfite; (2) oil-soluble antioxidants, such as ascorbyl palmitate, butylated hydroxyanisole (BHA), butylated hydroxytoluene (BHT), lecithin, propyl gallate and α tocopherol; and (3) metal chelating agents, such as citric acid, ethylenediamine tetraacetic acid (EDTA), sorbitol, tartaric acid and phosphoric acid.

Examples of pharmaceutically acceptable disintegrants include, but are not limited to: starch; clay; cellulose; alginate; gum; crosslinked polymers, such as crospolyvinylpyrrolidone or crospovidone (e.g., POLYPLASDONE XL from ISP (International Specialty Products, Wayne, N.J.)), croscarmellose sodium (e.g., AC-DI-SOL from FMC) and croscarmellose calcium; soybean polysaccharide; and guar gum.

Examples of pharmaceutically acceptable binders include, but are not limited to: starch; cellulose and derivatives thereof, such as microcrystalline cellulose (e.g., AVICEL PH from FMC (Philadelphia, Pa.), and hydroxypropyl cellulose, hydroxyethyl cellulose and hydroxypropyl methyl cellulose METHOCEL from Dow Chemical Corp., Midland, Mich.); sucrose; dextrose; corn syrup; polysaccharide; and gelatin.

Examples of pharmaceutically acceptable lubricants and pharmaceutically acceptable glidants include, but are not limited to: silica gel, magnesium trisilicate, starch, talc, tricalcium phosphate, magnesium stearate, aluminum stearate, calcium stearate, magnesium carbonate, magnesium oxide, polyethylene glycol, powdered cellulose, and microcrystalline cellulose.

Examples of pharmaceutically acceptable fillers and pharmaceutically acceptable diluents include, but are not limited to: powdered sugar, compressible sugar, glucose binding agents, dextrin, dextrose, lactose, mannitol, microcrystalline cellulose, powdered cellulose, sorbitol, sucrose and talc.

The optimal dose of each combination partner for treatment of cancer can be determined empirically for each individual using known methods and will depend upon a variety of factors, including, but not limited to: the degree of progression of the disease; the age, body weight, general health, gender and diet of the individual; the time and route of administration; and other medications the individual is taking. Optimal doses may be established using routine testing and procedures that are well known in the art.

The amount of each combination partner that may be combined with the carrier materials to produce a single dosage form will vary depending upon the individual treated and the particular mode of administration. In some embodiments, the unit dosage forms containing the combination of agents as described herein will contain a certain amount of each agent of the combination that is typically administered when the agent is administered alone.

In certain aspects, the pharmaceutical composition disclosed herein is used for treating or preventing cancer or for preparing a medicament for treating or preventing cancer. In a particular embodiment, the pharmaceutical combination disclosed herein is used for treating cancer or for preparing a medicament for treating cancer.

In some embodiments, the drug-carrier complex disclosed herein is administered to a patient in the form of a pharmaceutical composition. In some embodiments, the drug-carrier complex disclosed herein is present in a pharmaceutically effective amount.

In some embodiments, the cancer is selected from testicular cancer, ovarian cancer, cervical cancer, bladder cancer, osteosarcoma, head and neck cancer, small and non-small cell lung cancer, melanoma, lymphoma and pancreatic cancer. In some embodiments, the cancer is testicular cancer. In some embodiments, the cancer is ovarian cancer. In some embodiments, the cancer is cervical cancer. In some embodiments, the cancer is osteosarcoma. In some embodiments, the cancer is small cell lung cancer. In some embodiments, the cancer is non-small cell lung cancer. In some embodiments, the cancer is melanoma. In some embodiments, the cancer is lymphoma. In some embodiments, the cancer is pancreatic cancer.

The term “administered in combination with” or “co-administration” as used herein refers to the simultaneous or separate sequential administration in any manner of a solid or liquid oral pharmaceutical dosage form containing the drug-carrier complex disclosed herein and one or more other active agents known to be useful in the treatment of cancer, including chemotherapy and radiotherapy. The term “other one or more active agent” as used herein includes any compound or therapeutic agent known or proven to exhibit advantageous properties when administered to a patient in need of cancer treatment. As used herein, “other one or more active agents” is used interchangeably with other one or more anti-cancer drugs. Preferably, the compounds are administered in a close time proximity to each other if the administration is not simultaneous. Furthermore, it does not matter if the compounds are administered in the same dosage form. For example, one compound may be administered via injection and another compound may be administered orally.

In general, any antineoplastic agent showing activity against the treated susceptible tumor can be co-administered in the cancer treatment of the present invention. Examples of such agents can be found in Cancer—Principles and Practice of Oncology, Devita et al. (eds.), 6th edition, Feb. 15, 2001, Lippincott Williams & Wilkins Publishers. One of ordinary skill in the art would be able to discern which combination of agents would be useful based on the particular characteristics of the drugs and the disease involved.

Typical anti-cancer drugs for use in the present invention include, but are not limited to, β-lapachone, alkylating agents and nitrogen mustards and preparations thereof, mitomycin and preparations thereof, dihydrofolate reductase inhibitors and preparations thereof, thymidine synthase inhibitors and preparations thereof, purine nucleoside synthase inhibitors and preparations thereof, ribonucleotide reductase and inhibitors thereof, DNA polymerase inhibitors and preparations thereof, topoisomerase I inhibitors and preparations thereof, drugs that interfere with tubulin synthesis in mitosis (M) phase and preparations thereof, antineoplastic hormonal drugs and preparations thereof, biological response modifiers and preparations thereof, cyclin-dependent kinase inhibitors and preparations thereof, multi-target tyrosinase inhibitors and preparations thereof, antineoplastic biotin drugs and preparations thereof, cell differentiation inducers and preparations thereof, apoptosis inducers and preparations thereof, neoangiogenesis inhibitors and preparations thereof, EGFR inhibitors, extracts from traditional Chinese medicine for adjuvant therapy and preparations thereof, anti-epidermal growth factor receptor monoclonal antibodies, recombinant human granulocyte stimulating factor injections and preparations thereof, prostatic stem cell antigen antibodies, immunosuppressant drugs and preparations thereof, and PD-1/PD-L1 inhibitors. β-lapachone has a structural formula of

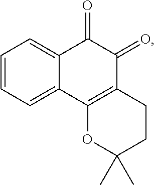 [SMILES: CC1(C)CCC2=C(O1)c1ccccc1C(=O)C2=O]

and it is a natural product isolated from the Lapacho trees in South American rainforest and kills a wide range of cancer cells in an NQO1 dependent manner (see Bey et al., Proc. Natl. Acad. Sci. U.S.A., 104:11832-11837 (2007)). In cancer cells that overexpress NQO1, β-lapachone undergoes inefficient oxidation reduction cycle, resulting in rapid, large-scale production of reactive oxygen (see Reinicke et al., Clin. Cancer Res., 11:3055-3064 (2005)).

Alkylating agents are non-phase specific anti-cancer agents and strong electrophiles. Typically, alkylating agents form covalent linkages, by alkylation, to DNA through nucleophilic moieties of the DNA molecule such as phosphate, amino, sulfhydryl, hydroxyl, carboxyl and imidazole groups. Such alkylation disrupts nucleic acid function, leading to cell death. Examples of alkylating agents include, but are not limited to, nitrogen mustards such as cyclophosphamide, melphalan and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as injection or tablet CYTOXAN®. Cyclophosphamide is indicated for the treatment of malignant lymphoma, multiple myeloma and leukemia as a single agent or in combination with other chemotherapeutic agents. Alopecia, nausea, vomiting and leukopenia are the most common dose limiting side effects of cyclophosphamide.

Melphalan, 4-[bis(2-chloroethyl)amino]-L-phenylalanine, is commercially available as an injection or tablet ALKERAN®. Melphalan is indicated for palliative treatment of multiple myeloma and non-resectable ovarian epithelial cancer. Myelosuppression is the most common dose limiting side effect of melphalan.

Chlorambucil, 4-[bis(2-chloroethyl)amino]benzenebutanoic acid, is commercially available as a tablet LEUKERAN®. Chlorambucil is indicated for palliative treatment of chronic lymphocytic leukemia, malignant lymphomas such as lymphosarcoma, giant follicular lymphoma, and Hodgkin's disease. Myelosuppression is the most common dose limiting side effect of chlorambucil.

Busulfan, 1,4-butanediol dimethanesulfonate, is commercially available as a tablet MYLERAN®. Busulfan is indicated for palliative treatment of chronic myelogenous leukemia. Myelosuppression is the most common dose limiting side effect of busulfan.

Carmustine, 1,3-[bis(2-chloroethyl)-1-nitrosourea, is commercially available as BiCNU® in the form of single vials of lyophilized material. Carmustine is indicated for palliative treatment of brain tumors, multiple myeloma, Hodgkin's disease and non-Hodgkin's lymphoma as a single agent or in combination with other agents. Delayed myelosuppression is the most common dose limiting side effect of carmustine.

Dacarbazine, 5-(3,3-dimethyl-1-triazeno)-imidazole-4-carboxamide, is commercially available as DTIC-Dome® in the form of single vials of material. Dacarbazine is indicated for the treatment of metastatic malignant melanoma and for, in combination with other agents, second-line treatment of Hodgkin's disease. Nausea, vomiting and anorexia are the most common dose limiting side effects of dacarbazine.

Mitomycin, [1aR-(1aα,8β,8aα,8bα)]-6-Amino-8-[[(aminocarbonyl)oxy]methyl]-1a,2, 8,8a,8b-hexahydro-8a-methoxy-5-methyl azirino [2′,3′:3,4]pyrrolo [1,2 Chemical book-α]indole-4,7-dione, also known as mitomycin C, is an effective antineoplastic agent in wide use today, and it is mainly used in the treatment of various solid tumors, such as gastric cancer, colon cancer, liver cancer, pancreatic cancer, non-small cell lung cancer, breast cancer and cancerous hydrothorax and ascites. It has bone marrow toxicity and can cause leucopenia and thrombopenia and result in phlebitis, and its presence outside the blood veins may cause tissue necrosis, alopecia, asthenia and liver and kidney function damage.

Antimetabolite antineoplastic agents are phase-specific antineoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite antineoplastic agents include, but are not limited to, thymidine synthase inhibitors (e.g., fluorouracil), dihydrofolate reductase inhibitors (e.g., methotrexate and pemetrexed), purine nucleotide synthase inhibitors (e.g., 6-mercaptopurine and thioguanine) and DNA polymerase inhibitors (e.g., cytarabine and gemcitabine).

5-fluorouracil, 5-fluoro-2,4-(1H,3H) pyrimidinedione, is commercially available as fluorouracil. Administration of 5-fluorouracil leads to inhibition of thymidylate synthesis and incorporation into both RNA and DNA. The result typically is cell death. 5-fluorouracil is indicated for the treatment of breast cancer, colon cancer, rectal cancer, gastric cancer and pancreatic cancers as a single agent or in combination with other chemotherapeutic agents. Other fluoropyrimidine analogs include 5-fluoro deoxyuridine (floxuridine) and 5-fluorodeoxyuridine monophosphate.

Cytarabine, 4-amino-1-β-D-arabinofuranosyl-2(1H)-pyrimidinone, is commercially available as CYTOSAR-U® and is commonly known as Ara-C. It is believed that cytarabine exhibits cell cycle specificity at S phase by inhibiting DNA chain elongation by terminal incorporation of cytarabine into the growing DNA chain. Cytarabine is indicated for the treatment of acute leukemia as a single agent or in combination with other chemotherapeutic agents. Other cytidine analogs include 5-azacytidine and 2′,2′-difluorodeoxycytidine (gemcitabine).

Mercaptopurine, 1,7-dihydro-6H-purine-6-thione monohydrate, is commercially available as PURINETHOL®. Mercaptopurine exhibits cell cycle specificity at S phase by inhibiting DNA synthesis through an unspecified mechanism. Mercaptopurine is indicated for the treatment of acute leukemia as a single agent or in combination with other chemotherapeutic agents. A useful mercaptopurine analog is azathioprine.

Thioguanine, 2-amino-1,7-dihydro-6H-purine-6-thione, is commercially available as TABLOID®. Thioguanine exhibits cell cycle specificity at S phase by inhibiting DNA synthesis through an unspecified mechanism. Thioguanine is indicated for the treatment of acute leukemia as a single agent or in combination with other chemotherapeutic agents. Other purine analogs include pentostatin, erythrohydroxynonyladenine, fludarabine phosphate and cladribine.

Gemcitabine, 2′-deoxy-2′,2′-difluorocytidine monohydrochloride (β-isomer), is commercially available as GEMZAR®. Gemcitabine exhibits cell cycle specificity at S phase by blocking progression of cells through the G1/S boundary. Gemcitabine is indicated for the treatment of locally advanced non-small cell lung cancer in combination with cisplatin and for the treatment of locally advanced pancreatic cancer as a single agent.

Methotrexate, N-[4[[(2,4-diamino-6-pteridinyl)methyl]methylamino]benzoyl]-L-glutamic acid, is commercially available as methotrexate sodium. Methotrexate exhibits cell cycle specificity at S phase by inhibiting DNA synthesis, repair and/or replication through the inhibition of dihydrofolate reductase which is required for synthesis of purine nucleotides and thymidylate. Methotrexate is indicated for the treatment of choriocarcinoma, meningeal leukemia, non-Hodgkin's lymphoma, breast cancer, head cancer, neck cancer, ovarian cancer and bladder cancer as a single agent or in combination with other chemotherapeutic agents.

Ribonucleotide reductase inhibitors are inhibitors of transcription or translation of genes encoding ribonucleotide reductase. Compounds (such as hydroxyurea) inhibit the activity of ribonucleotide reductase by causing the destruction of the tyrosyl radical through disruption of the iron core of the protein R2 (McClarty et al., 1990) and thereby preventing cells from passing through the S phase of the cell cycle (Ashihara and Baserga, 1979).

Anti-microtubule agents or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids derived from natural sources are phase specific anti-cancer agents that operate at the G2/M phases of the cell cycle. It is believed that the diterpenoids stabilize the β-tubulin subunit of the microtubules by binding to this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2β,4,7β,10β,13α-hexahydroxytax-11-en-9-one-4,10-diacetate-2-benzoate-13-ester with (2R,3S)-N-benzoyl-3-phenylisoserine, is a natural diterpene product isolated from the Pacific yew tree Taxus brevifolia and is commercially available as an injection TAXOL®. It is a member of the taxane family of terpenes. Paclitaxel has been approved for clinical use in the treatment of refractory ovarian cancer and breast cancer in the United States.

Docetaxel, (2R,3S)-N-carboxy-3-phenylisoserine,N-tert-butyl ester,13-ester with 5β-20-epoxy-1,2α,4,7β,10β,13α-hexahydroxytax-11-en-9-one 4-acetate-2-benzoate trihydrate, is commercially available as injection TAXOTERE®. Docetaxel is indicated for the treatment of breast cancer. Docetaxel is a semisynthetic derivative of paclitaxel q.v., and it is prepared using a natural precursor, 10-deacetyl-baccatin III, extracted from the needle of the European Yew tree. The dose limiting toxicity of docetaxel is neutropenia.

Vinca alkaloids are phase specific antineoplastic agents derived from the periwinkle plant. Vinca alkaloids act at the M phase (mitosis) of the cell cycle by specifically binding to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine and vinorelbine.

Vinblastine, vincaleukoblastine sulfate, is commercially available as an injection VELBAN®. Although it is possibly indicated for second-line treatment of various solid tumors, it is primarily indicated for the treatment of testicular cancer and various lymphomas including Hodgkin's disease, lymphocytic lymphoma and histiocytic lymphoma. Myelosuppression is a dose limiting side effect of vinblastine.

Vincristine, 22-oxovincaleukoblastine, is commercially available as an injection ONCOVIN®. Vincristine is indicated for the treatment of acute leukemia, and has also been found to be useful in treatment regimens for Hodgkin's and non-Hodgkin's malignant lymphomas. Alopecia and nervous system effects are the most common side effects of vincristine, with myelosuppression and gastrointestinal mucositis effects occurring to a lesser extent.

Vinorelbine, 3′,4′-didehydro-4′-deoxy-C′-norvincaleukoblastine[R-(R*,R*)-2,3-dihydroxybutanedioate(1:2)(salt)], is commercially available as an injection of vinorelbine tartrate (NAVELBINE®) and is a semisynthetic vinca alkaloid. Vinorelbine is indicated for the treatment of various solid tumors, particularly non-small cell lung, advanced breast cancer and hormone refractory prostate cancer, as a single agent or in combination with other chemotherapeutic agents, such as cisplatin. Myelosuppression is the most common dose limiting side effect of vinorelbine.

Several protein tyrosine kinases catalyze the phosphorylation of specific tyrosyl residues in a variety of proteins involved in the regulation of cell growth. These protein tyrosine kinases can be broadly classified as receptor or non-receptor kinases.

Receptor tyrosine kinases are transmembrane proteins with an extracellular ligand binding domain, a transmembrane domain and a tyrosine kinase domain. Receptor tyrosine kinases are involved in the regulation of cell growth and are generally referred to as growth factor receptors. Inappropriate or uncontrolled activation of many of these kinases, i.e., aberrant kinase growth factor receptor activity, such as overexpression or mutation, has been shown to result in uncontrolled cell growth. Thus, the aberrant activity of these kinases is associated with malignant tissue growth. Therefore, inhibitors of such kinases can provide methods for cancer treatment. Growth factor receptors include, for example, epidermal growth factor receptor (EGFr), platelet derived growth factor receptor (PDGFr), erbB2, erbB4, ret, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptor, Trk receptors (TrkA, TrkB and TrkC), ephrin (eph) receptor, and RET protooncogene. Several growth receptor inhibitors are under development and include ligand antagonists, antibodies, tyrosine kinase inhibitors and antisense oligonucleotides. Growth factor receptors and agents that inhibit growth factor receptor function are described, for example, in Kath et al., Exp. Opin. Ther. Patents, 10(6):803-818 (2000); and Lofts et al., “Growth factor receptors as targets”, New Molecular Targets for Cancer Chemotherapy, Workman et al. (eds.), CRC Press, London, 1994.

Tyrosine kinases that are not growth factor receptor kinases are referred to as non-receptor tyrosine kinases. Non-receptor tyrosine kinases used in the present invention are targets or potential targets for anti-cancer agents and include cSrc, Lck, Fyn, Yes, Jak, cAbl, FAK (focal adhesion kinase), Bruton's tyrosine kinase and Bcr-Abl. Such non-receptor kinases and agents that inhibit the function of non-receptor tyrosine kinases are described in Sinh et al., J Hemat & Stem Cell Res., 8(5):465-480 (1999); and Bolen et al., Annual Rev. Immuno, 15:371-404 (1997).

Exemplary multi-target tyrosinase inhibitors include, for example, sorafenib, regorafenib and sunitinib.

Antibiotic antineoplastic agents are non-phase specific agents that bind to or are inserted into DNA. Typically, such action results in stable DNA compounds or strand breakage, which disrupts the normal function of the nucleic acid, leading to cell death. Examples of antibiotic antineoplastic agents include, but are not limited to, actinomycins such as dactinomycin, anthracyclines such as daunomycin and doxorubicin; and bleomycin.

Dactinomycin, also known as actinomycin D, is commercially available as an injectable form. Dactinomycin is indicated for the treatment of Wilm's tumor and rhabdomyosarcoma. Nausea, vomiting and anorexia are the most common dose limiting side effects of dactinomycin.

Daunomycin, (8S-cis-)-8-acetyl-10-[(3-amino-2,3,6-trideoxy-α-L-lyxo-pyransoyl)oxy]-7,8,9,10-tetrahydro-6,8,11-trihydroxy-1-methoxy-5,12naphthacenedione hydrochloride, is commercially available as a liposomal injectable form or as an injection. Daunomycin is indicated for remission induction in the treatment of acute non-lymphocytic leukemia and advanced HIV-associated Kaposi's sarcoma. Myelosuppression is the most common dose limiting side effect of daunomycin.

Doxorubicin, (8S,10S)-10-[(3-amino-2,3,6-trideoxy-α-L-lyxo-hexopyranosyl)oxy]-8-hydroxyacetyl,7,8,9,10-tetrahydro-6,8,11-trihydroxy-1-methoxy-5,12-naphthacenedione hydrochloride, is commercially available as an injectable form or ADRIAMYCIN®. Doxorubicin is primarily indicated for the treatment of acute lymphoblastic leukemia and acute myeloid leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available. Bleomycin is indicated for palliative treatment of squamous cell carcinoma, lymphoma and testicular cancer as a single agent or in combination with other agents. Pulmonary and cutaneous toxicities are the most common dose limiting side effects of bleomycin.

Topoisomerase I inhibitors include, but are not limited to, topotecan, irinotecan, 9-nitrocamptothecin, and the macromolecular camptothecin conjugate PNU-166148 (compound A1 in WO99/17804). Irinotecan can be administered, for example, in a commercially available form, such as under the trademark CAMPTOSAR®. Topotecan can be administered, for example, in a commercially available form, such as under the trademark HYCAMTIN®.

Cell differentiation inducers are agents used for reversing immature malignant cells and differentiating them into normal cells in inducing differentiation of tumors. Exemplary cell differentiation inducers include retinoids and vitamin D3, phenylacetate and its analog phenylbutyrate, interferon, arsenious acid, and the like.

Apoptosis inducers are capable of causing regression of tumors by inducing apoptosis of tumor cells or by inducing apoptosis of vascular endothelial cells of tumors. Common apoptosis inducers include hormones (e.g., dexamethasone), cytokines (e.g., IL-2, TGF-β, IL-10 or IFN-T), antibodies (e.g., anti-IgM antibody, anti-IgD antibody or anti-HLA-II antibody), superantigens (e.g., SPE), intracellular signaling molecule modulators (e.g., cycloheximide), and the like.

Anti-tumor hormones and hormonal analogs are useful compounds for treating cancer, and there is a relationship between the hormone and the growth and/or lack of growth of tumor. Examples of hormones and hormonal analogues useful in cancer treatment include, but are not limited to, adrenocorticosteroids such as prednisone and prednisolone useful in the treatment of malignant lymphoma and acute leukemia in children; aminoglutethimide and other aromatase inhibitors such as anastrozole, letrazole, vorozole and exemestane useful in the treatment of adrenocortical carcinoma and hormone dependent breast carcinoma containing estrogen receptors; progesterons such as megestrol acetate useful in the treatment of hormone dependent breast cancer and endometrial carcinoma; estrogens, androgens and anti-androgens such as flutamide, nilutamide, bicalutamide, cyproterone acetate and 5α-reductases such as finasteride and dutasteride, useful in the treatment of prostatic cancer and benign prostatic hypertrophy; anti-estrogens such as tamoxifen, toremifene, raloxifene, droloxifene, idoxifene, as well as selective estrogen receptor modulators (SERMS) such those described in U.S. Pat. Nos. 5,681,835, 5,877,219 and 6,207,716, useful in the treatment of hormone dependent breast cancer and other susceptible cancers; and gonadotropin-releasing hormone (GnRH) and analogues thereof which stimulate the release of leutinizing hormone (LH) and/or follicle stimulating hormone (FSH) to treat prostatic cancer, such as LHRH agonists and antagonists, e.g., goserelin acetate and leuprolide.

Biological response modifiers include, but are not limited to, BCG vaccine (sold under the tradenames theraCys® and TICE® BCG) and denileukin diftitox (sold under the tradename Ontak®).

Cell cycle signaling inhibitors inhibit molecules involved in cell cycle control. A family of protein kinases known as cyclin-dependent kinases (CDKs) and their interaction with a family of proteins known as cyclins control progression through the eukaryotic cell cycle. Coordinated activation and inactivation of different cyclins/CDK complexes is necessary for normal progression through the cell cycle. Several cell cycle signaling inhibitors are under development. Examples of cyclin-dependent kinases (including CDK2, CDK4 and CDK6) and inhibitors thereof are described, for example, in Rosania et al., Exp. Opin. Ther. Patents, 10(2):215-230 (2000). Furthermore, p21WAF1/CIP1 has been described as a potent and versatile inhibitor of cyclin-dependent kinases (CDKs) (Ball et al., Progress in Cell Cycle Res., 3:125 (1997)). Compounds that induce expression of p21WAF1/CIP1 are known to be involved in cell proliferation inhibition and have tumor-inhibiting activity (Richon et al., Proc. Nat. Acad. Sci. USA., 97(18):10014-10019 (2000)), and are incorporated as cell cycle signaling inhibitors. Histone deacetylase (HDAC) inhibitors are involved in the transcriptional activation of p21WAF1/CIP1 (Vigushin et al., Anticancer Drugs, 13(1):1-13 (2002)) and are suitable cell cycle signaling inhibitors used herein.

Neoangiogenesis inhibitors include, but are not limited to, non-receptor MEK angiogenesis inhibitors. Anti-angiogenic agents such as those that inhibit the action of vascular endothelial growth factor (e.g., the anti-vascular endothelial cell growth factor antibody bevacizumab Avastin®) and compounds that work by other mechanisms (e.g., tricarboxyaminoquinoline, inhibitors of integrin αvβ3 function, endostatin and angiostatin).

Epidermal growth factor receptor (EGFR) inhibitors include, but are not limited to, gefitnib (sold under the tradename Iressa®), N-[4-[(3-chloro-4-fluorophenyl)amino]-7-[[(3″S″)-tetrahydro-3-furanyl]oxy]-6-quinazolinyl]-4(dimethylamino)-2-butenamide sold under the tradename Tovok® by Boehringer Ingelheim), cetuximab (sold under the tradename Erbitux® by Bristol-Myers Squibb), and panitumumab (sold under the tradename Vectibix® by Amgen).

Exemplary anti-epidermal growth factor receptor (EGFR) monoclonal antibodies include, but are not limited to: cetuximab (Erbitux), panitumumab, matuzumab (EMD-72000), trastuzumab, nimotuzumab (hR3), zalutumumab, TheraCIMh-R3, MDX0447 (CAS339151-96-1), and ch806 (mAb-806, CAS946414-09-1).

Extracts from traditional Chinese medicine for adjuvant therapy of cancer include, but are not limited to, ginsenoside Rh2, curcumin, Herba Scutellariae Barbatae extract, radix astragali, berberine, etc.

Prostatic stem cell antigen antibodies are antibodies that can bind to proteins related to prostatic stem cell antigen (PSCA). Exemplary PSCA antibody drugs comprise, for example, HERCEPTIN® and RITUXANO® (both from Genetech), which have been successfully used to treat breast cancer and non-Hodgkin's lymphoma, respectively.

Classes of immunosuppressant drugs include, but are not limited to, calcineurin inhibitors, such as cyclosporin A or FK 506; mTOR inhibitors, such as rapamycin, 40-O-(2-hydroxyethyl)-rapamycin, CCI779, ABT578 or AP23573; ascomycins with immunosuppressive properties, such as ABT-281 or ASM981; corticosteroid; leflunomide; mizoribine; mycophenolic acid; mycophenolate mofetil; 15-deoxyspergualin or immunosuppressive homologs, analogs or derivatives thereof; immunosuppressive monoclonal antibodies, e.g., monoclonal antibodies for leukocyte receptors, such as MHC, CD2, CD3, CD4, CD7, CD8, CD25, CD28, CD40, CD45, CD58, CD80 or CD86 or ligands thereof; other immunomodulatory compounds, e.g., recombinant binding molecules having at least a portion of the extracellular domain of CTLA4 or mutants thereof, e.g., at least an extracellular portion of CTLA4 or mutants thereof bound to a non-CTLA4 protein sequence, e.g., CTLA4 Ig (e.g., designated ATCC 68629) or mutants thereof, e.g., LEA29Y; adhesion molecule inhibitors, such as LFA-1 antagonists, ICAM-1 or -3 antagonists, VCAM-4 antagonists or VLA-4 antagonists. PD1 inhibitors include nivolumab (also known as MDX-1106, MDX-1106-04, ONO-4538 or BMS0936558; CAS accession No.: 946414-94-4) disclosed in, for example, U.S. Pat. No. 8,008,449; pembrolizumab (also known as lambrolizumab, MK-3475, MK03475, SCH-900475 or KEYTRUDA) disclosed in, for example, U.S. Pat. No. 8,354,509 and WO 2009/114335; immunoadhesin (e.g., an immunoadhesin comprising an extracellular or PD-1 binding portion of PD-L1 or PD-L2 fused to a constant region (e.g., the Fc region of an immunoglobulin sequence)); pidilizumab (CT-011; Cure Tech), a humanized IgG1k monoclonal antibody that binds to PD1 (pidilizumab and other humanized anti-PD-1 monoclonal antibodies are disclosed in WO 2009/101611); AMP-224 (B7-DCIg; Amplimmune) disclosed in WO2010/027827 and WO2011/066342, a PD-L2Fc fusion soluble receptor that blocks the interaction between PD1 and B7-H1; and other inhibitors of PD-1, such as anti-PD1 antibodies disclosed in U.S. patent application Ser. Nos. 8,609,089, 2010028330 and/or 20120114649.

PD-L1 inhibitors include: MSB0010718C (also known as A09-246-2; Merck Serono), a monoclonal antibody that binds to PD-L1 and is disclosed, for example, in WO 2013/0179174; an anti-PD-L1 binding antagonist selected from YW243.55.S70 and MPDL3280A (Genetech/Roche), a human Fc optimized IgG1 monoclonal antibody that binds to PD-L1 (MDPL3280A and other human monoclonal antibodies for PD-L1 are disclosed in U.S. Pat. No. 7,943,743 and U.S. Patent Publication No. 20120039906); and MEDI-4736, MSB-0010718C or MDX-1105 (MDX-1105, also known as BMS-936559, is an anti-PD-L1 antibody described in WO 2007/005874; antibody YW243.55.S70 is an anti-PD-L1 antibody described in WO 2010/077634).

### Embodiments

Embodiments of the present invention will be described in detail below, and the examples of the embodiments are illustrated in the accompanying drawings. The embodiments described below by reference to the accompanying drawings are exemplary and are merely intended to illustrate the present invention, and are not to be construed as limiting the present invention. Unless otherwise stated, in the embodiments, HSA refers to albumin, and GEM (Gem) refers to gemcitabine.

The drug protein ratio (DPR) in the present invention represents the molar ratio of the platinum-based drug loaded on the albumin to the albumin in the platinum-based drug-albumin compound, and is slightly smaller than the feeding molar ratio of the platinum-based drug to the albumin when preparing the platinum-based drug-albumin compound.

In addition, in the present application, cisplatin was taken as an example of a platinum-based drug, a drug-carrier complex of the platinum-based drug and albumin was prepared, and the formed drug-carrier complex was characterized and evaluated for performance. The methods for forming the drug-carrier complexs using other platinum-based drugs and albumin and performing characterization and performance evaluation on the formed drug-carrier complexs are basically the same as those for cisplatin.

Meanwhile, in the present application, pancreatic cancer was taken as an example, and detailed experimental studies were performed on the anti-cancer effects (such as targeting property, therapeutic effect and drug safety) of the above drug-carrier complex. The method for examining the anti-cancer effect for other cancers (such as lung cancer) is almost the same as that for pancreatic cancer, and the above drug-carrier complex has the same anti-cancer effect for other cancer as that for pancreatic cancer.

### 1. Methods and Materials

### 1.1. Preparation of Cisplatin-Albumin Compound

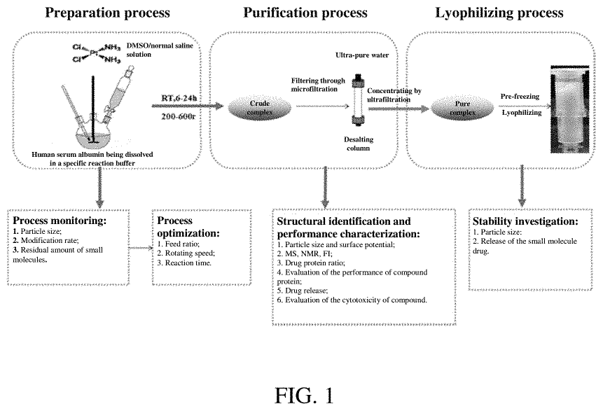

500 mg of human serum albumin was dissolved in a reaction buffer, and the prepared human serum albumin solution was placed in a reaction bottle with a stirring device (the rotating speed should not be too high) for stabilization for 10-20 min. Cisplatin powder with a corresponding mass was dissolved in saline solution, and then the solution of cisplatin in saline solution was added dropwise into the human serum albumin solution. The rotating speed remained unchanged, and the mixture was reacted at room temperature for 8 h to obtain a crude product of cisplatin-albumin compound. The preparation process of cisplatin-albumin compound is shown in FIG. 1.

### 1.2. Purification and Characterization of Cisplatin-Albumin Compound

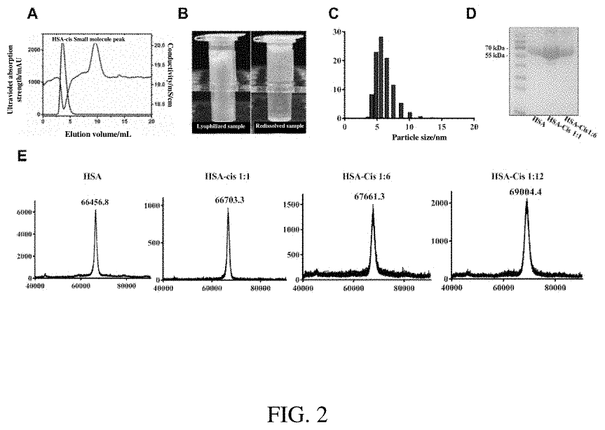

The crude product of cisplatin-albumin compound obtained in the step 1.1 was filtered using an aqueous microporous filter membrane with a pore size of 0.45 micrometers. The crude compound was purified using a fast protein purification system to remove unbound cisplatin and other small molecules that are not suitable for injection. The purification conditions were as follows: purification device: ÄKTA™ Purifier 100; desalting column: Hitrap Desalting 5 mL columns connected in series; eluent: deionized water. After loading, a separated product under the first UV280 absorption peak was collected, and the purified cisplatin-albumin compound was obtained (as shown in FIG. 2A). The pre-frozen product of the purified cisplatin-albumin compound was lyophilized using a lyophilizer, thus obtaining lyophilized powder of the purified cisplatin-albumin compound. The purified cisplatin-albumin compound was characterized for the following: particle size, electrophoretic analysis and mass spectrometry. Meanwhile, the release of cisplatin of the cisplatin-albumin compound in a phosphate buffer was measured using HPLC to evaluate the stability of the cisplatin-albumin compound under physiological conditions.

### 1.3. Evaluation of In Vitro Cytotoxicity and Cell Migration of Cisplatin-Albumin Compound

Human pancreatic cancer cell strains PANC-1 and MIA PaCa-2 (the cell sources were all ATCC) were used, and the culture conditions were as follows: DMEM high-glucose medium containing 10% fetal bovine serum, 1% non-essential amino acid and 100 units of penicillin and streptomycin. Cells in the growth period were taken, and then the cells in each experimental group were digested with pancreatin and resuspended in a complete culture medium to obtain a single cell suspension. The cells were counted, and the seeding density for the cytotoxicity experiment was set as 5000 cells/well (a 96-well plate). PANC-1 and MIA PaCa-2 cells were each treated with the cisplatin-albumin compound according to the set concentration, and CCK8 was added 48 h after the compound was added. The absorbance value was read at 405 nm, and the inhibition rates of the cisplatin-albumin compound for PANC-1 and MIA PaCa-2 were calculated, wherein, cell viability=absolute absorbance value of experimental group×100%/absolute absorbance value of control group.

In the cell migration experiment, PANC-1 cells were used, cell density was set to be 50000 cells/well (a 6-well plate), and a DMEM high-glucose medium contained 10% fetal bovine serum and penicillin and streptomycin at a certain concentration. The cells were incubated in an incubator at 37° C./5% CO₂, 3 duplicate wells were set for each group, and the culture system was 400 μL/well. The medium was replaced with low concentration serum medium (containing 1% FBS) for cell culturing when the confluence of cells reached 90% or more. A 1 mL pipette tip was vertically aimed to the central part of the lower end of the 6-well plate, and was then slightly pushed upwards to form a scratch. The plate was gently rinsed 2-3 times with PBS, and a low-concentration serum culture medium containing cisplatin at a specific concentration and one containing cisplatin-albumin compound at a specific concentration were added to the wells. A picture was taken at 0 h. The mixture was incubated in an incubator at 37° C./5% CO₂, and a proper time was decided according to healing degree to scan the plate with a cell imaging device. Migration area and migration rate were analyzed and calculated. Migration area=cell area at 24 h−cell area at 0 h; migration rate=migration area/cell area at 0 h; migration index=migration rate of compound treatment group/migration rate of control group.

### 1.4. Evaluation of PK and Tumor-Targeting Property of Cisplatin-Albumin Compound

Mouse pancreatic cancer cells transfected with luciferase reporter genes were used to prepare mouse pancreatic cancer orthotopic transplantation models. The tumor volume of the mice were determined by injecting fluorescein sodium into tail veins, and the mice with similar tumor volume were selected for evaluation of systemic PK and tumor-targeting property. Mice were administered, via injection at the tail veins, with cisplatin (8 mg/kg) and cisplatin-albumin compound 6:1 (HSA-Cis 1:6, the effective dose of cisplatin in the compound is 8 mg/kg, and the dose of albumin HSA is about 320 mg/kg), and then the mice were sacrificed at 0.5 h, 3 h, 6 h, 12 h, 24 h and 48 h, and plasma, heart, liver, spleen, lung, kidney and pancreatic cancer orthotopic tumor were taken. Pt content in plasma and organs was measured by ICP-MS, and the tissue distribution of Pt in mice was calculated, thereby evaluating the targeting property of cisplatin-albumin compound for tumor tissues. Pharmacokinetic parameters were calculated using PK Solver 2.0.

### 1.5. Evaluation of Anti-Tumor Effect of Cisplatin-Albumin Compound In Vivo

Tumor-bearing BALB/c Nude mouse models were used for evaluating the therapeutic effect of the cisplatin-albumin compound on subcutaneous transplantation tumor models of human pancreatic cancer cell strain PANC-1. The human pancreatic cancer cell strain PANC-1 was cultured in a DMEM high-glucose medium containing 10% FBS. Before being used, the cells were digested with pancreatin and centrifuged for 3 min at 800 r·min⁻¹ to remove the supernatant. The cells were resuspended in a serum-free DMEM medium, matrigel was added at a ratio of 1:1, and the number of the cells was adjusted to 5×10⁶/mL. The cell suspension was inoculated subcutaneously to the right thigh at 100 μL/mouse. The mean tumor size reached 50-100 mm³ on day 10 after the inoculation. The tumor-bearing mice were randomly divided into 5 groups with 6 mice in each. The groups were as follows: PBS control group (PBS), cisplatin group (cisplatin, 4 mg/kg), cisplatin-albumin compound 1:1 group (HSA-Cis 1:1, wherein the effective dose of cisplatin in the compound was 4 mg/kg, and the dose of albumin was about 960 mg/kg), cisplatin-albumin compound 6:1 group (HSA-Cis 1:6, wherein the effective dose of cisplatin in the compound was 4 mg/kg, and the dose of albumin was 160 mg/kg), and cisplatin-albumin compound 12:1 group (HSA-Cis 1:12, wherein the dose of cisplatin in the compound was 4 mg/kg, and the dose of albumin was about 80 mg/kg). The drugs were administered via tail vein injection. The administration was performed once a week, 4 times in total. The long (l) and short (r) diameters of the tumors were measured with a vernier caliper every other day to calculate tumor volume. All mice were sacrificed on day 11 after the administration cycle was completed, and they were dissected to get the tumors, which were then weighed. The tumor volume calculation formula was as follows: V=(l×r²)/2.

### 1.6. Kaplan-Meier Survival Curves

Pancreas of BALB/c nude mice was transplanted orthotopically with Mia paca-2 cells (reporter gene: luciferase) to prepare pancreatic cancer transplantation in situ models, and then the mice were randomly divided into 4 groups with 6 mice in each, and administered with drugs 10 days after the models were established. The administration information was as follows: PBS control group (PBS), cisplatin group (cisplatin, 5 mg/kg), cisplatin-albumin compound 6:1 medium dose group (HSA-Cis 1:6, 5 mg/kg, wherein the effective dose of cisplatin in the compound was 5 mg/kg and the dose of HSA was 200 mg/kg), and cisplatin-albumin compound 6:1 low dose group (HSA-Cis 1:6, 1 mg/kg, wherein the effective dose of cisplatin in the compound was 1 mg/kg and the dose of HSA was 40 mg/kg). The drugs were administered once every 7 days, three times in total. The mice were weighed every other day, and the survival state of the mice was recorded. Kaplan-Meier survival curves were drawn to examine the influence of different drug treatments on the survival state of the mice.

### 1.7. Evaluation of Safety of Cisplatin-Albumin Compound

Maximum tolerated dose (MTD): healthy Balb/C mice were injected with a certain dose of cisplatin and cisplatin-albumin compound 6:1 in the tail veins, and the mice were recorded for changes in appearance, behavior, diet, secretion and excretion after administration. The MTD could be recorded as greater than this dose/kg if no animals died.

Blood biochemistry and liver renal function analysis: mice with pancreatic cancer PANC-1 subcutaneous transplantation tumor were administered according to the following doses: PBS control group (PBS), cisplatin group (cisplatin, 4 mg/kg), cisplatin-albumin compound 1:1 group (HSA-Cis 1:1, wherein the effective dose of cisplatin in the compound was 4 mg/kg, and the dose of HSA was 960 mg/kg), cisplatin-albumin compound 6:1 group (HSA-Cis 1:6, wherein the effective dose of cisplatin in the compound was 4 mg/kg, and the dose of HSA was 160 mg/kg), and cisplatin-albumin compound 12:1 group (HSA-Cis 1:12, wherein the effective dose of cisplatin in the compound was 4 mg/kg, and the dose of HSA was 80 mg/kg). 11 days after the administration cycle was completed, whole blood and liver were taken for blood cell analysis, and myelosuppression toxicity and hematotoxicity of the cisplatin-albumin compound were evaluated based on the trend of blood cells (it is generally believed that granulocytopenia typically begins one week after the withdrawal of chemotherapeutic drug and reaches a minimum level by 10-14 days of withdrawal, and in this study, mice were dissected 10-11 days after withdrawal of drug treatment.)

1.8. Preparation and Performance Evaluation of Compounds Formed by Other Platinum-Based Drugs such as Carboplatin, Nedaplatin and Oxaliplatin with Albumin

A proper amount of human serum albumin was dissolved in a reaction buffer, and the prepared human serum albumin solution was placed in a reaction bottle with a stirring device (the rotating speed should not be too high) for stabilization for a period of time. Carboplatin, nedaplatin and oxaliplatin powders with a corresponding mass were each dissolved in a corresponding solvent, and then the carboplatin, nedaplatin and oxaliplatin solutions were each added into the human serum albumin solution. The rotating speed remained unchanged, and the mixtures each reacted at room temperature for a certain period of time to obtain crude products of carboplatin-, nedaplatin- and oxaliplatin-albumin compounds. The crude products of carboplatin-, nedaplatin- and oxaliplatin-albumin compounds above were each filtered using an aqueous microporous filter membrane with a pore size of 0.45 micrometers. The crude compounds were purified using a fast protein purification system to remove unbound carboplatin, nedaplatin, oxaliplatin and other small molecules that are not suitable for injection, thus obtaining purified cisplatin-albumin compound. The purified carboplatin-, nedaplatin- and oxaliplatin-albumin compounds were lyophilized, thus obtaining lyophilized powders of carboplatin-, nedaplatin- and oxaliplatin-albumin compounds. The purified carboplatin-, nedaplatin- and oxaliplatin-albumin compounds were subjected to characterization and performance evaluation for the following: particle size, electrophoretic analysis, mass spectrometry, NMR structural analysis, stability analysis, cytotoxicity evaluation and anti-tumor effect evaluation. Each characterization method or performance evaluation method was the same as that for cisplatin-albumin compound.

### 2. Results and Analysis

### 2.1. Preparation and Characterization of Cisplatin-Albumin Compound

### 2.1.1. Specific Preparation and Characterization of Cisplatin-Albumin Compound

Cisplatin-albumin compound was prepared according to the flow chart shown in FIG. 1. Its purification spectrum of fast protein purification system is shown in FIG. 2A. In the figure, HSA-Cis was an ultraviolet absorption peak at UV280, and was a sample peak of the cisplatin-albumin compound; the small molecule peak was a conductance change curve of a sample in the purification process, and separation of the cisplatin-albumin compound from unbound cisplatin and small molecule substances which were not suitable for injection in a reaction buffer was realized through desalting columns connected in series. The dried powder of the lyophilized cisplatin-albumin compound was white powder (as shown in the left centrifuge tube of FIG. 2B), and a clear cisplatin-albumin compound solution (as shown in the right centrifuge tube of FIG. 2B) could be obtained by redissolving the lyophilized cisplatin-albumin compound with a PBS solution. The particle size of the cisplatin-albumin compound was measured using a dynamic light scattering particle size analyzer, and the results are shown in FIG. 2C. The average particle size (number average particle size) of the cisplatin-albumin compound was 3-10 nm, which was the same as the particle size of albumin, indicating that the cisplatin-albumin compound existed in the form of a protein monomer. The SDS-PAGE electrophoresis image of the cisplatin-albumin compound is shown in FIG. 2D, which shows that the cisplatin-albumin compound existed mainly as a protein monomer and its molecular weight was close to that of albumin, and the molecular weight of the compound slightly increased along with the increase of the cisplatin in the cisplatin-albumin compound. The results of mass spectrometry analysis of albumin and cisplatin-albumin compound are shown in FIG. 2E. According to calculation, it was found that the drug protein ratio (DPR) of cisplatin-albumin compound 1:1 group HSA-Cis (1:1) was 0.91, the DPR of cisplatin-albumin compound 6:1 group HSA-Cis (1:6) was 5.43, and the DPR of cisplatin-albumin compound 3:1 group HSA-Cis (1:3) was 11.2, indicating that there is strong binding ability between cisplatin molecule and albumin, and the albumin-bound cisplatin molecule increases gradually with the increase of the feed ratio. The release amount of cisplatin micromolecules of the molecular cisplatin-albumin compound in a phosphate buffer with a pH of 7.4 was smaller than 10%, as detected quantitatively by HPLC, which indicated that the cisplatin-albumin compound has strong stability under physiological conditions; meanwhile, the cisplatin-albumin compound had significantly improved release rate in a phosphate buffer with a pH of 5.0.

2.1.2. Performance Comparison of Cisplatin-Albumin Compounds with Different Feed Ratios

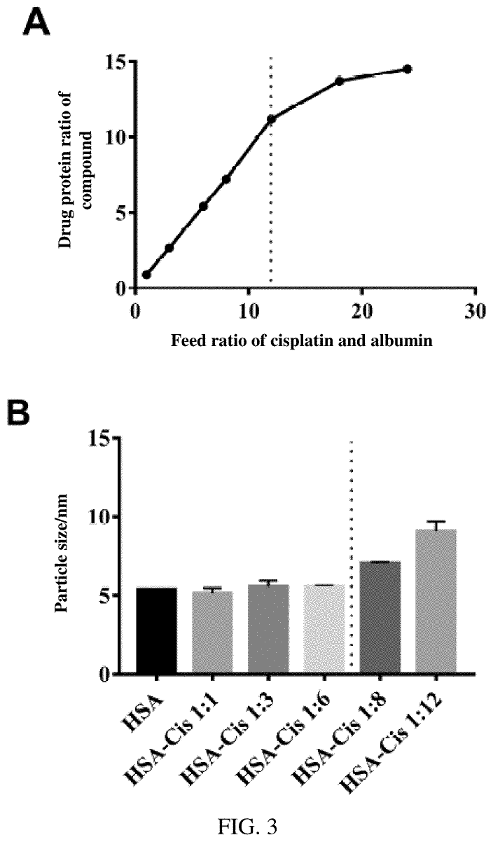

Performance comparison of cisplatin-albumin compounds with different feed ratios is shown in FIG. 3. In the figure, A shows the change trend of the drug protein ratio (drug-to-protein molar ratio) of the purified compound along with the feed amount of cisplatin. As can be seen from the figure, when the feed ratio of cisplatin to albumin was smaller than 12, DPR increased linearly with the increase of feed ratio; the DPR increase trend was slightly flat when the feed ratio was greater than 12, which indicates that the sites of the albumin molecules for binding to cisplatin tend to be saturated and cannot bind to more drug molecules. B shows the particle size of the cisplatin-albumin compounds with different feed ratios. When the feed ratio was smaller than 6, the particle size of the obtained compound was close to that of the albumin; when the feed ratio was greater than 8, the particle size of the compound significantly increased, and a large number of large particles appeared in the compound reaction system as the feed ratio increased. For example, the average particle size of the reaction system was 180 nm at a feed ratio of 18 (results not shown) and 326 nm at a feed ratio of 24.

### 2.2. Evaluation of In Vitro Cytotoxicity and Cell Migration of Cisplatin-Albumin Compound

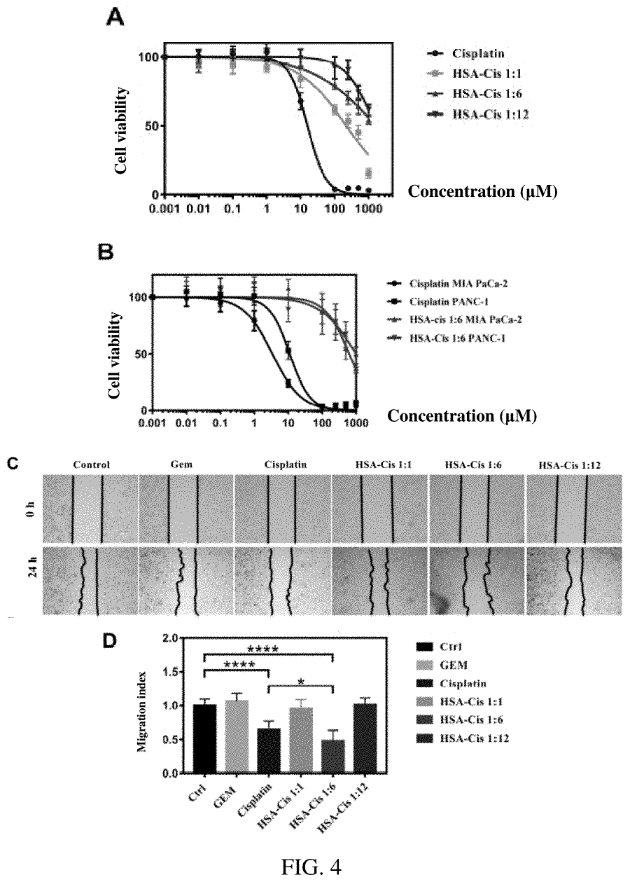

The tumor cytotoxicity and cell migration inhibitory effect of the cisplatin-albumin compound are shown in FIG. 4. The cell proliferation inhibition data in FIG. 4A revealed the following results: firstly, the cisplatin-albumin compound changes the cellular entry pathway of cisplatin, so that the total amount of platinum-based drug entering cells in a certain time period is lower than that of cisplatin, and therefore, the cisplatin-albumin compound is lower in cytotoxicity than cisplatin from what is shown, which is closely related to the cellular entry efficiency and the cellular entry pathway. Secondly, cisplatin-albumin compounds with different DPRs have differences in cytotoxicity to human pancreatic cancer cell strain MIA PaCa-2. The osmotic pressure inside and outside cells is changed due to high protein concentration in a low DPR system, so that cell necrosis is easily caused, and high cytotoxicity is thereby present (which indicates that the cisplatin-albumin compound with low DPR possibly has high risk in administration). Thirdly, it was found from cytotoxicity studies that cisplatin-albumin compounds with relatively high DPR (DPR>5) have similar effect on inhibition of cell proliferation (n=5). The results in FIG. 4B show that the cytotoxicity results of cisplatin and cisplatin-albumin compound (HSA-cis 1:6 group) for another human pancreatic cancer cell strain PANC-1 are similar to the results for MIA PaCa-2. Compared with direct cellular entry of cisplatin small molecules, the cellular entry pathway of the compound molecules is mainly energy-dependent endocytosis pathway so that the amount of drug entering the cells in the same time is smaller than that of cisplatin group. Therefore, the cytotoxicity of the compound for human pancreatic cancer cell strains MIA PaCa-2 and PANC-1 is lower than that of cisplatin (n=5), wherein n=5 represents 5 duplicate wells.

The results of cisplatin and cisplatin-albumin compounds with different DPRs against tumor cell migration are shown in FIGS. 4C and 4D. As can be seen from the qualitative (4C) and quantitative (4D) results, the inhibition rates of tumor cell migration of cisplatin-albumin compounds with different DPRs are different, wherein the migration rate of PANC-1 cells treated with the compound of the cisplatin-albumin compound 6:1 group is the lowest, as shown in the quantitative analysis results of cell migration experiment of FIG. 4D.

### 2.3. Evaluation of PK and Tumor-Targeting Property of Cisplatin-Albumin Compound

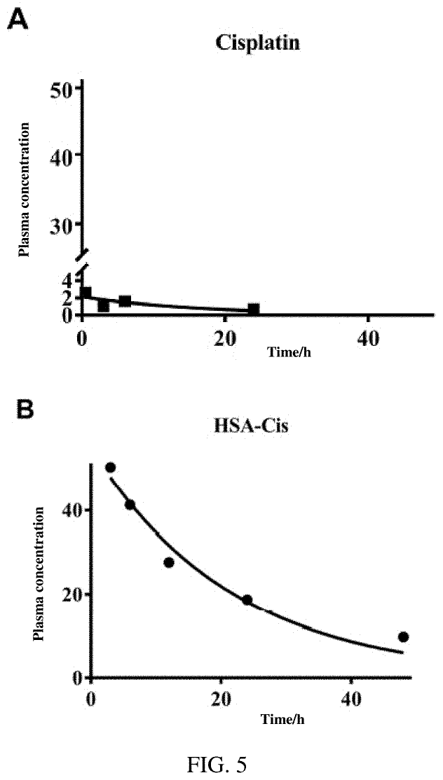

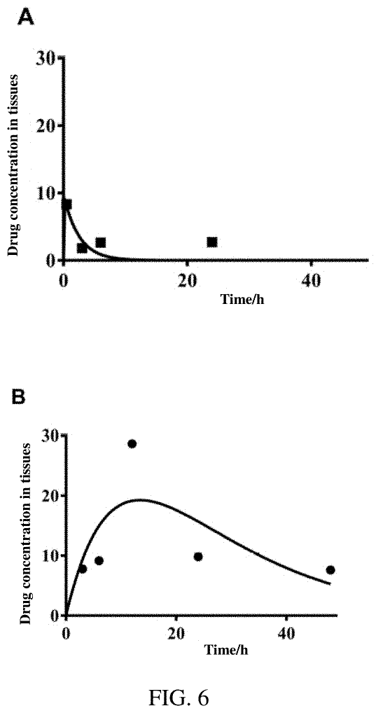

Based on literature research, pancreatic cancer orthotopic transplantation model mice (reporter gene: luciferase) were administered, via injection at the tail veins, with cisplatin (8 mg/kg) and cisplatin-albumin compound 6:1 (HSA-Cis, wherein the effective dose of cisplatin is 8 mg/kg), and then the mice were sacrificed at 0.5 h, 3 h, 6 h, 12 h, 24 h and 48 h, and plasma, heart, liver, spleen, lung, kidney and pancreatic cancer orthotopic tumor were taken. Pt content in plasma and organs was measured by ICP-MS, and pharmacokinetic profiles of cisplatin and cisplatin-albumin compound in plasma and at pancreatic orthotopic tumor regions in mice were simulated and calculated using PKslvor2.0, and the results are shown in FIGS. 5 and 6 and Tables 1 and 2.

TABLE 1
Pharmacokinetic parameters of cisplatin
and cisplatin-albumin compound in plasma

| Parameters | t_{1/2} | V | CL | AUC₀₋ₜ | AUC_{0-inf} | AUMC | MRT | Vss |
|---|---|---|---|---|---|---|---|---|
| Cisplatin | 11.41 | 3.72 | 0.23 | 27.17 | 35.41 | 582.80 | 16.46 | 3.72 |
| HAS-Cis | 15.00 | 0.15 | 0.01 | 1063.11 | 1192.83 | 25806.18 | 21.63 | 0.15 |

From the plasma pharmacokinetic profile of cisplatin group in FIG. 5A and the plasma pharmacokinetic profile of cisplatin-albumin compound in FIG. 5B (pharmacokinetic parameters are shown in Table 1 above), it can be seen that the plasma pharmacokinetics of cisplatin-albumin compound in vivo, compared with cisplatin, has the following characteristics:

1. the Pt content of the cisplatin-albumin compound in the plasma at the same time point is 30 times or more higher than that of the cisplatin, and this indicates that the blood circulation time of the cisplatin-albumin compound is prolonged, which is probably because a lot of drugs in the compound are still tightly bound with albumin, while only a small part of cisplatin molecules in free cisplatin are still in the plasma, and most of drug molecules have been eliminated or have entered into peripheral cells;
2. compared with the AUC of the cisplatin group, the AUC of the cisplatin-albumin compound is significantly improved.

TABLE 2
Pharmacokinetic parameters of cisplatin and cisplatin-albumin compound in tumor tissues

| Parameters | t_{1/2}ka | t_{1/2}k10 | V/F | CL/F | Tmax | Cmax | AUC₀₋ₜ | AUC_{0-inf} | AUMC | MRT |
|---|---|---|---|---|---|---|---|---|---|---|
| Cisplatin | 0.02 | 1.73 | 84.32 | 33.79 | 0.13 | 9.30 | 24.41 | 24.41 | 61.60 | 2.52 |
| HAS-Cis | 8.65 | 10.04 | 16.94 | 1.17 | 13.43 | 19.27 | 613.81 | 705.33 | 19013.96 | 26.96 |

By comparing the pharmacokinetic data (as shown in Table 2) of cisplatin-albumin compound (B in FIG. 6) and cisplatin (FIG. 6A) at the tumor site in tumor-bearing mice, it can be found that:
1. the cisplatin-albumin compound can significantly improve the maximum drug concentration (Cmax) of the drug at a tumor site;
2. the cisplatin-albumin compound can effectively prolong the accumulation time of the drug at a tumor site (the t_{1/2}ka of cisplatin-albumin compound is 8.65 h, and the t_{1/2}ka of cisplatin is 0.02 h);
3. the cisplatin-albumin compound can significantly improve the AUC of the drug's accumulation at a tumor site (the AUC of cisplatin-albumin compound is 705.33, and the AUC of cisplatin is 24.41).

By combining the results in Tables 1 and 2, it is shown that the cisplatin-albumin compound has the following advantages compared with cisplatin:

1. the cisplatin-albumin compound can significantly improve the targeting property of Pt at a tumor site; the ID% of the HSA-Cis group can reach more than 25% while the ID% of the cisplatin group is less than 5% according to the calculation formula ID%=drug amount at a tumor site×100%/injected drug amount;
2. the cisplatin-albumin compound can effectively improve the drug concentration of Pt in tumor tissues, and the Cmax of the cisplatin-albumin compound group is 2-3 times that of the cisplatin group; in addition, compared with the cisplatin group, the mean retention time MRT of Pt of the cisplatin-albumin compound group in the tumor tissues can be improved by 10 times or more;
3. by stable binding of Pt with albumin, the cisplatin-albumin compound can effectively improve the tissue distribution of Pt in a mouse body, effectively increase the accumulation concentration of Pt-based cytotoxic drugs in a tumor area, and thereby reduce the distribution of Pt in normal tissues and alleviate the toxicity of the Pt-based drugs.

### 2.4. Evaluation of Anti-Tumor Effect of Cisplatin-Albumin Compound

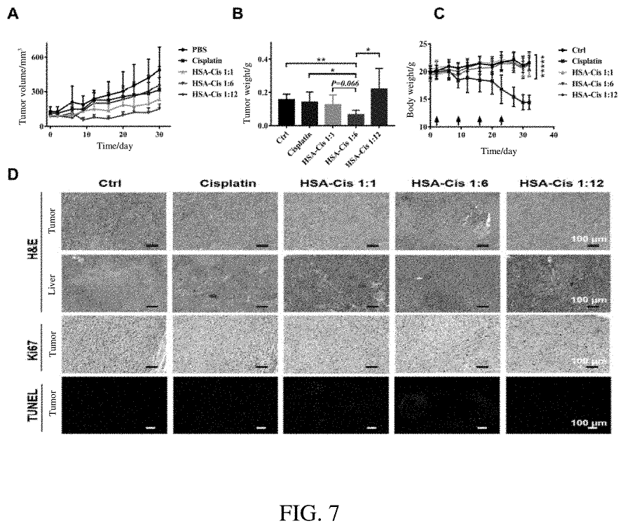

In order to evaluate the effect of cisplatin-albumin compound in inhibiting tumor volume increase in the pancreatic cancer subcutaneous transplantation tumor models, and to evaluate whether different drug modification rates (DPRs) affect the effect of cisplatin-albumin compound in inhibiting tumor volume increase, the inventors carried out specific tests and the results are shown in FIG. 7. The DPRs of the cisplatin-albumin compound 1:1 group, the cisplatin-albumin compound 6:1 group and the cisplatin-albumin compound 12:1 group are as follows in sequence: HSA-Cis 1:1 DPR=0.9, HSA-Cis 1:6 DPR=5.4, and HSA-Cis 1:12 DPR=11.2 (data calculated from mass spectrometry results). The tumor volume change curves for PANC-1 subcutaneous transplantation tumor in FIG. 7A and the tumor weight results in FIG. 7B show that:

free cisplatin (4 mg/kg) has no significant inhibition effect on PANC-1 subcutaneous transplantation tumor; the cisplatin-albumin compound 12:1 has no significant inhibition effect on PANC-1 subcutaneous transplantation tumor, and this is supposed to be related to the rapid metabolism of the cisplatin-albumin compound 12:1 in vivo. Compared with the PBS control group and cisplatin control group, the cisplatin-albumin compound 1:1 and the cisplatin-albumin compound 6:1 have significant inhibition effect on PANC-1 subcutaneous transplantation tumor, wherein the cisplatin-albumin compound 6:1 exhibits the best tumor-inhibition effect.

The body weight change curve of the mice during the administration process is shown in FIG. 7C, wherein in the cisplatin group, after administration, the feeding ability of the mice was remarkably reduced, the body weight of the mice remarkably decreased during the administration process, and the survival state of the mice was extremely poor during the treatment process. In the cisplatin-albumin compound group, whether the DPR was high or low, the tolerance of the mice was better after administration, the body weight change was not significant, and the survival state of the mice was good in the treatment process.

To further analyze the tumor-inhibiting mechanism for tumor tissues and the degree of damage to normal organs in mice after administration in the cisplatin group and cisplatin-albumin compound groups, HE staining analysis was performed on tumor tissues and liver tissues of the treatment groups, and the results are shown in the upper two rows of FIG. 7D. As can be seen from HE staining data, the tumor progression of the tumor tissues of the cisplatin-albumin compound 6:1 group is inhibited, and compared with other groups, the cisplatin-albumin compound 6:1 group has the advantages of minimal liver damage, smooth and complete liver surface and no obvious liver damage. The liver of cisplatin group had extremely many relatively large perforation holes. In addition, significant liver damage occurred in the cisplatin-albumin compound 1:1 group and the cisplatin-albumin compound 12:1 group. In the 1:1 group, there were numerous proliferative neoplasms in the liver tissue, while in the 12:1 group, both perforation of the liver and abnormal hyperplasia of the liver were observed. This result suggests that DPR is a key factor in the balance of therapeutic effect and toxicity effect of the cisplatin-albumin compound, and that a proper DPR can be effective in exerting efficacy and controlling toxicity.

To further investigate the mechanism of action of cisplatin-albumin compound in inhibiting tumor growth, Ki67 and TUNEL immunohistochemical analysis was also performed on tumor tissues after administration, and the results are shown in the lower two rows of FIG. 7D. From the Ki67 results, it is found that in the cisplatin-albumin compound 6:1 group, the cell proliferation of the tumor tissues is remarkably lower than that of groups of cisplatin and cisplatin-albumin compounds with other different DPRs; TUNEL immunohistochemical analysis also shows that in the cisplatin-albumin compound 6:1 group, apoptosis of tumor cells is higher than that in other groups. In conclusion, a cisplatin-albumin compound with a proper DPR can effectively inhibit the proliferation of tumor cells and promote the apoptosis of the tumor cells, thereby controlling the growth of tumors.

### 2.5. Kaplan-Meier Survival Curve of Cisplatin-Albumin Compound

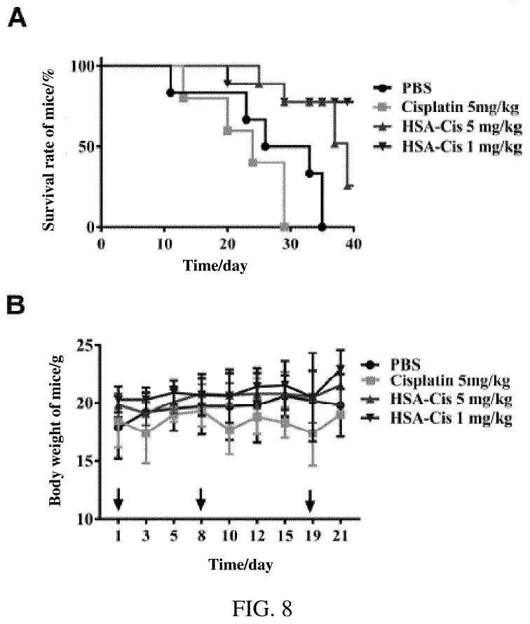

The evaluation results of the therapeutic effect of the cisplatin-albumin compound (6:1 group) on the MIA PaCa-2 pancreas orthotopic transplantation models are shown in FIG. 8. FIG. 8A shows the Kaplan-Meier survival curves of cisplatin and cisplatin-albumin compound (6:1 group) after administration via the tail veins, and the results show that the survival advantage of the cisplatin-albumin compound group is greater than that of the cisplatin group (P<0.05), and the survival advantage of the low-dose cisplatin-albumin compound group is significantly higher than that of other experimental groups. By referring to PK and tissue distribution data analysis in section 2.3, cisplatin-albumin complexation can increase the drug concentration and retention time of Pt in tumor tissues and increase the metabolic burden on the liver as well. Therefore, an ideal treatment mode is to increase the concentration of the tumor drug and meanwhile maintain or reduce the drug concentration in the liver. The low-dose compound group can better achieve the effect, and thus the survival time of the mice is remarkably prolonged, and the survival quality of the animals is improved. The body weight change curve of the mice in the whole treatment process is shown in FIG. 8B. After administration, the mice in the cisplatin group were in a poor feeding state, and the body weight was significantly reduced. After administration, the mice in the cisplatin-albumin compound groups were in a good feeding and survival state, and the total body weight was not significantly reduced. Even if the body weight of the mice in the compound group slightly decreased after the administration, the mice rapidly recovered after the end of the administration. The above results show that the toxicity of the cisplatin-albumin compounds is lower than that of chemotherapeutic drug cisplatin, and a better therapeutic effect can be achieved if the administration dose of the cisplatin-albumin compounds is appropriately reduced.

### 2.6. Evaluation of Safety of Cisplatin-Albumin Compound

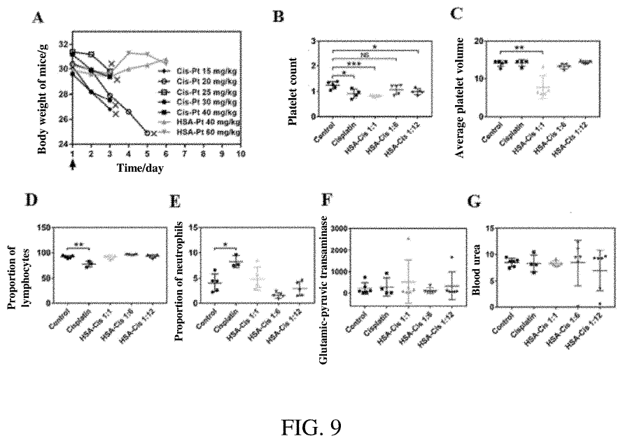

The maximum tolerated doses of cisplatin-albumin compound (6:1 group) and cisplatin group are shown in FIG. 9A, and it can be seen from the results that after administration with 40 mg/kg (dose of cisplatin in compound) and 60 mg/kg (dose of cisplatin in compound) of cisplatin-albumin compounds (6:1 group), the mice all survived, and they gained weight and survived well. In the cisplatin groups of 15 mg/kg, 20 mg/kg, 25 mg/kg, 30 mg/kg and 40 mg/kg, the body weight of mice was significantly reduced after administration, and all the animals in these experimental groups died. From the above results, it can be seen that the MTD of the cisplatin-albumin compound groups is>60 mg/kg (the dose of cisplatin in compound), and the MTD of the cisplatin group is<15 mg/kg. The results show that the cisplatin-albumin compound groups can significantly improve the acute toxicity of cisplatin and remarkably improve the maximum tolerated dose of cisplatin.

FIGS. 9B-9E are the results of blood cell analysis 11 days after administration of cisplatin-albumin compounds and cisplatin. FIG. 9B shows the results of platelet count 11 days after the end of administration. From the statistical results, it can be seen that after the end of administration, compared with the control group, the number of platelets in the cisplatin-albumin compound 6:1 group was not significantly reduced, while the number of platelets was reduced in cisplatin group and the other two cisplatin-albumin compound groups with different DPRs (1:1 group and 12:1 group). The results have statistical significance, indicating that in the cisplatin group and the cisplatin-albumin compound groups with two different DPRs, a certain degree of damage to the hematopoietic system of mice was caused after administration; the cisplatin-albumin compound group (6:1 group) has low blood toxicity as its results of platelets after administration are similar to those of the control group. According to the analysis of the change of the average platelet volume in FIG. 9C, the compound with higher protein concentration (group 1:1) can affect the blood coagulation function of the body as the too high protein concentration changes the body osmotic pressure and thereby causes the irreversible reduction of the platelet volume. The analysis of lymphocytes and neutrophils in FIGS. 9D and 9E shows that after administration, the cisplatin group exhibited a down-regulated lymphocyte fraction and an increased neutrophil fraction, indicating a certain tendency towards myelosuppression, whereas the compound group showed no significant tendency towards myelosuppression. FIG. 9F shows blood biochemical analysis results for liver function 11 days after administration of cisplatin-albumin compounds and cisplatin, and none of the compound groups showed significant liver function damage after administration, wherein the ordinate unit is U/L. FIG. 9G shows blood biochemical analysis results for renal function 11 days after administration of cisplatin-albumin compounds and cisplatin, and no significant change in the renal function was observed after the end of the administration cycle compared with the control group, wherein the ordinate unit is mmol/L.

2.7. Preparation and Performance Evaluation of Compounds Formed by Other Platinum-Based Drugs such as Carboplatin, Nedaplatin, Oxaliplatin and Lobaplatin with Albumin

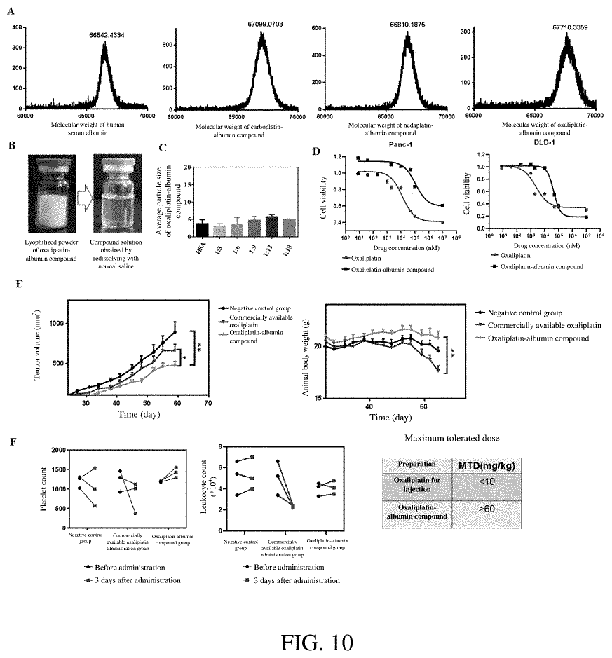

Carboplatin-, nedaplatin- and oxaliplatin-albumin compounds were prepared according to the flow chart shown in FIG. 1, and the schematic diagram of the mass spectrometry results of the carboplatin-, nedaplatin- and oxaliplatin-albumin compounds obtained after purification by the fast protein purification system is shown in FIG. 10A. The molecular weight of the human serum albumin, as measured by Maldi-tof, is 66542, and the molecular weights of the carboplatin-, nedaplatin- and oxaliplatin-albumin compounds prepared according to the embodiment are 67099, 66810 and 67710, respectively. The mass spectrum characterization results show that the molecular weights of the stable compounds formed by carboplatin, nedaplatin and oxaliplatin with albumin are significantly greater than those of human serum albumin. Besides, according to the previous results about cisplatin and subsequent analysis results of the molecular weights of carboplatin, nedaplatin and oxaliplatin, the molecular weight of the compound will increase gradually along with the increase of feed amount of platinum-based drug.

The dried powder of lyophilized oxaliplatin-albumin compound was white powder (as shown in a vial on the left in FIG. 10B), and a clear oxaliplatin-albumin compound solution (as shown in a vial on the right in FIG. 10B) could be obtained by redissolving the lyophilized oxaliplatin-albumin compound with a saline solution solution. The particle size of the oxaliplatin-albumin compound was measured using a dynamic light scattering particle size analyzer, and the results show that the average particle size (number average particle size) of the oxaliplatin-albumin compound was 3-10 nm, which was the same as the particle size of albumin, indicating that the oxaliplatin-albumin compound existed in the form of a protein monomer. FIG. 10C shows the particle size of the resulting oxaliplatin-albumin compounds with different feed ratios. The particle size of the compound increases gradually with the increase of the feed ratio, but is close to the particle size of albumin. This is different from the cisplatin-albumin system researched previously, and the reason lies in the different chemical structures of cisplatin and oxaliplatin. A cisplatin molecule has four active groups and is extremely easy to be mutually crosslinked in albumin molecules and among albumin molecules, so that the particle size of a cisplatin compound remarkably increases along with the increase of the feed amount, and the active groups in the oxaliplatin structure are relatively fewer, so that the crosslinking is not easily formed among the albumin molecules, and the particle size change is relatively stable.

FIG. 10D shows the evaluation of the cell proliferation inhibitory effect of oxaliplatin-albumin compound against human pancreatic cancer cell strain Panc-1 (FIG. 10D, left side) and human colon cancer cell strain DLD-1 (FIG. 10D, right side) (n=5). From the results, it can be seen that the inhibition rate of oxaliplatin-albumin compound against tumor cells is high. In addition, due to the difference in the cellular entry pathways of oxaliplatin and oxaliplatin-albumin compound, the cytotoxicity of the compound on human tumor cell strains is lower than that of a free drug group. The increase of multiple in IC50 for the oxaliplatin-albumin compound preparation is less than that for the cisplatin-albumin compound, which is presumably due to structural difference in the oxaliplatin-albumin compound and the cisplatin-albumin compound. Because the amino in the oxaliplatin is protected, the number of multi-coordination molecules in the oxaliplatin-albumin compound is small, and the drug is easy to release in cells.

In order to evaluate the effect of oxaliplatin-albumin compound in inhibiting tumor volume increase in the pancreatic cancer subcutaneous transplantation tumor models, a test was carried out and the results are shown in FIG. 10E. The experimental groups were negative control group (glucose injection), commercial available oxaliplatin preparation group and oxaliplatin-albumin compound group. FIG. 10E shows, on the left, the curves of volume change in PANC-1 subcutaneous transplantation tumor of the negative control group (glucose injection), commercial available oxaliplatin preparation group and oxaliplatin-albumin compound group. The results show that commercially available oxaliplatin preparation at 5 mg/kg has no significant inhibiting effect on PANC-1 subcutaneous transplantation tumor; compared with the commercially available oxaliplatin injection, the oxaliplatin-albumin compound preparation with the same dose has better tumor proliferation inhibition effect on PANC-1 subcutaneous transplantation tumor. As can be seen from the change in body weight of mice after administration (FIG. 10E, right side): compared with the free oxaliplatin preparation control group, the body weight change of the mice in the oxaliplatin-albumin compound group was not significant after administration, while the body weight of the mice in the commercial available oxaliplatin preparation group was rapidly reduced after administration. Based on the change trend of the body weight of mice after administration, it can be seen that the toxicity of the oxaliplatin-albumin compound preparation is effectively controlled, and meanwhile, the anti-tumor effect of the oxaliplatin-albumin compound preparation is enhanced, which shows that the design of the oxaliplatin-albumin compound preparation features higher rationality and clinical application value.

FIG. 10F shows the results of primary blood cell analysis before and 3 days after administration of oxaliplatin-albumin compound and commercially available oxaliplatin preparation, and the maximum tolerated dose of oxaliplatin-albumin compound and commercially available oxaliplatin preparation after 4 consecutive administrations. The left graph in FIG. 10F shows the counting results of platelets before and 3 days after administration, and it can be seen from the statistical results that the number of platelets in the oxaliplatin-albumin compound group after the end of administration was not significantly reduced compared with the control group, while the number of platelets in the commercially available oxaliplatin preparation group was reduced. The results have statistical significance, indicating that in the commercially available oxaliplatin preparation group, a certain degree of damage to the hematopoietic system of mice was caused after administration, and the oxaliplatin-albumin compound has relatively low blood toxicity as there was no significant difference in the platelets after administration between the oxaliplatin-albumin compound group and the control group. The graphs in FIG. 10F show the counting results of mouse leukocytes before and 3 days after administration. The analysis shows that the proportion of the leukocytes of the commercially available oxaliplatin preparation group was remarkably reduced after administration, and the level of leukocytes of the oxaliplatin-albumin compound group was not remarkably different from that in the negative control group after administration.

The maximum tolerated doses of oxaliplatin-albumin compound and commercially available oxaliplatin preparation are shown on the right in FIG. 10F, and after 4 administrations, the mice in the oxaliplatin-albumin compound 83.4 mg/kg and 60 mg/kg groups, all survived and were in good survival state although the body weight of the mice decreased by about 5-10%. After 4 administrations, the body weight of mice in the commercially available oxaliplatin preparation 10 mg/kg, 15 mg/kg and 25 mg/kg groups was significantly reduced, more than half of the mice in the 10 mg/kg group died, and all animals in the other two dose groups died. From the above results, it can be seen that the MTD of the oxaliplatin-albumin compound group is>60 mg/kg, and the MTD of the commercially available oxaliplatin preparation group is<10 mg/kg. The results show that the oxaliplatin-albumin compound groups can significantly improve the acute toxicity of oxaliplatin and remarkably improve the maximum tolerated dose of oxaliplatin.

1. A drug-carrier complex, comprising an active ingredient and a carrier, wherein the active ingredient is loaded on the carrier, the active ingredient is a platinum-based drug, and the carrier is albumin.

2. The drug-carrier complex according to claim 1, wherein the platinum-based drug is selected from cisplatin, carboplatin, oxaliplatin, nedaplatin, lobaplatin, picoplatin, satraplatin and triplatin.

3. The drug-carrier complex according to claim 2, wherein the platinum-based drug and the albumin carrier form a relatively stable protein-drug compound system via a bond selected from a covalent bond, a coordination bond and a non-covalent bond, the non-covalent bond being selected from a hydrogen bond, Van der Waals' force and hydrophobic interaction.

4. The drug-carrier complex according to claim 1, wherein the albumin is selected from serum-derived albumin, bioengineered recombinant albumin and analogs thereof.

5. The drug-carrier complex according to claim 4, wherein the serum-derived albumin is selected from human serum albumin and bovine serum albumin.

6. The drug-carrier complex according to claim 1, wherein the average particle size of the drug-carrier complex is 3-10 nm.

7. The drug-carrier complex according to claim 1, wherein the molar ratio of the platinum-based drug to the albumin is 0.5:1-24:1.

8. The drug-carrier complex according to claim 7, wherein the molar ratio of the platinum-based drug to the albumin is 2.5:1-11.2:1.

9. The drug-carrier complex according to claim 8, wherein the molar ratio of the platinum-based drug to the albumin is 5.0:1-7.0:1.

10. A method for preparing the drug-carrier complex according to claim 1, comprising:
subjecting the platinum-based drug and the albumin to a mixing-combining process in a specific buffer to obtain a mixture; and purifying the resulting mixture to obtain the drug-carrier complex.

11. The method according to claim 10, further comprising dispersing the platinum-based drug in a solvent prior to the mixing-combining, wherein the solvent is selected from saline solution, glucose injection, water for injection, phosphate buffer, DMSO and methanol.

12. The method according to claim 11, wherein the concentration of the platinum-based drug in saline solution is 0.1-20 mg/mL.

13. The method according to claim 10, wherein the albumin is dispersed in a buffer.

14. The method according to claim 13, wherein the concentration of the albumin is 5-50 mg/mL.

15. The method according to claim 13, wherein the buffer comprises 20-60 mmol/L phosphate, 10-200 mmol/L NaCl and 2-20 mmol/L EDTA.

16. The method according to claim 13, wherein the pH of the buffer is 4-10.

17. The method according to claim 11, wherein the mixing-combining is performed by adding a dispersion of the platinum-based drug dropwise to a buffer solution of the albumin.

18. The method according to claim 10, wherein the molar ratio of the platinum-based drug to the albumin is 1:1-1:40.

19. A pharmaceutical composition, comprising the drug-carrier complex according to claim 1 and at least one pharmaceutically acceptable carrier.

20. A method for treating a subject with cancer, comprising administrating a medicament comprising an effective amount of the drug-carrier complex according to claim 1 to the subject with cancer.

21. The method according to claim 20, wherein the cancer is selected from testicular cancer, ovarian cancer, cervical cancer, bladder cancer, osteosarcoma, head and neck cancer, small and non-small cell lung cancer, melanoma, lymphoma, lung cancer and pancreatic cancer.

22. The method according to claim 20, wherein the medicament is administrated in combination with at least one other anti-cancer drug.

23. The method according to claim 22, wherein the other anti-cancer drug is selected from β-lapachone, alkylating agents and nitrogen mustards and preparations thereof, mitomycin and preparations thereof, dihydrofolate reductase inhibitors and preparations thereof, thymidine synthase inhibitors and preparations thereof, purine nucleoside synthase inhibitors and preparations thereof, ribonucleotide reductase and inhibitors thereof, DNA polymerase inhibitors and preparations thereof, topoisomerase I inhibitors and preparations thereof, drugs that interfere with tubulin synthesis at mitosis M phase and preparations thereof, antineoplastic hormonal drugs and preparations thereof, biological response modifiers and preparations thereof, cyclin-dependent kinase inhibitors and preparations thereof, multi-target tyrosinase inhibitors and preparations thereof, antineoplastic biotin drugs and preparations thereof, cell differentiation inducers and preparations thereof, apoptosis inducers and preparations thereof, neoangiogenesis inhibitors and preparations thereof, EGFR inhibitors, extracts from traditional Chinese medicine for adjuvant therapy and preparations thereof, anti-epidermal growth factor receptor monoclonal antibodies, recombinant human granulocyte stimulating factor injections and preparations thereof, prostatic stem cell antigen antibodies, immunosuppressant drugs and preparations thereof, and PD-1/PD-L1 inhibitors.
